(19) United States
(12) Patent Application Publication
(10) Pub. No.: US 2002/0137891 A1
(43) Pub. Date: Sep. 26, 2002

(54) Protein scaffold and its use to multimerise monomeric polypeptides

(76) Inventors: Fergal Conan Hill, Les Martres de Veyre (FR); Jean Chatellier, Les Martres de Veyre (FR); Alan Roy Fersht, Cambridge (GB)

Correspondence Address: FROMMER LAWRENCE & HAUG, 745 FIFTH AVENUE- 10TH FL., NEW YORK, NY, 10151, US

(21) Appl. No.: 10/007,628

(22) Filed: Nov. 8, 2001

Related U.S. Application Data
(63) Continuation-in-part of application No. PCT/GB00/01815, filed on Dec. 5, 2000.

(30) Foreign Application Priority Data
May 14, 1999 (GB) 9911298.9
Dec. 3, 1999 (GB) 9928788.0
Dec. 6, 1999 (GB) 9928831.8

(51) Int. Cl. A61K 38/00; C07K 1/00; C07K 17/00; C07H 21/04; C07K 14/00

(52) U.S. Cl. 530/350; 514/12; 514/13; 514/14; 514/15; 514/21; 536/23.4

(57) ABSTRACT

The invention relates to polypeptide monomer capable of oligomerisation, said monomer comprising a heterologous amino acid sequence inserted into the sequence of a subunit of an oligomerisable protein scaffold.

The present invention provides a polypeptide scaffold which can be used to multimerise monomeric polypeptides or protein domains, to produce multimeric proteins having any desired characteristic. In particular, the invention relates to oligomerisable scaffolds, methods for producing oligomeric proteins comprising such scaffolds, and to oligomeric proteins comprising such scaffolds.

It is often desirable to multimerise polypeptide monomers. For example, although variable domains of antibodies, particularly when expressed as single chains (scFv), have many advantages associated with small size; however, their avidity in vivo is often disappointing, their half-life is limited and they are often unable to trigger biological responses. As observed by Libyh et al. (1997) Blood 90:3978, monomeric recombinant molecules prove generally unsatisfactory for in vivo use. Most biological systems are multivalent, either structurally, associating different chain, or functionally

Different approaches have been proposed, in the prior art, for the multimerisation of recombinant protein domains. For example, chemical linkage of proteins to polymers such as polyethylene glycol has been attempted (Katre et al., (1987) PNAS (USA) 84:1487). This technique, however, is cumbersome and requires large amounts of purified material. In antibody molecules, modifications of the disulphide-forming possibilities in the hinge, and other, regions of the molecules have been attempted in order to modulate the extent to which antibodies will associate with each other. Results, however, have been inconsistent and unpredictable. Similarly, use of protein A fusions to generate multimeric antibodies may successfully link antibody fragments, but is of limited application in other fields.

Libyh et al., (1997) Blood 90:3978, described a protein multimerisation system which is based on the c-terminal part of the α-chain of complement component 4 binding protein (C4bp). C4bp is involved in the regulation of the complement system. It is a multimeric protein comprising 7 identical alpha chains and a single beta chain. Using only a C-terminal fragment of C4bp, Libyh et al. were able to induce spontaneous multimerisation of associated antibody fragments to create homomultimers of scFv fragments. The portion of C4bp used was placed C-terminal to the scFv sequence, optionally spaced by a MYC tag.

Many proteins require the assistance of molecular chaperones in order to be fold in vivo or to be refolded in vitro in high yields. Molecular chaperones are proteins, which are often large and require an energy source such as ATP to function. A key molecular chaperone in Escherichia coli is GroEL, which consists of 14 subunits each of some 57.5 kD molecular mass arranged in two seven membered rings. There is a large cavity in the GroEL ring system, and it is widely believed that the cavity is required for successful protein folding activity. For optimal activity, a co-chaperone, GroES, is required which consists of a seven membered ring of 10 kD subunits. The activity of the GroEL/GroES complex requires energy source ATP. GroEL and GroES are widespread throughout all organisms, and often referred to as chaperonin (cpn) molecules, cpn 60 and cpn 10 respectively.

GroEL is an allosteric protein. Allosteric proteins are a special class of oligomeric proteins, which alternate between two or more different three-dimensional structures on the binding of ligands and substrates. Allosteric proteins are often involved in control processes in biology or where mechanical and physico-chemical energies are interconverted. The role of ATP is to trigger this allosteric change, causing GroEL to convert from a state that binds denatured proteins tightly to one that binds denatured proteins weakly. The co-chaperone, GroES, aids in this process by favouring the weak-binding state. It may also act as a cap, sealing off the cavity of GroEL. Further, its binding to GroEL is likely directly to compete with the binding of denatured substrates. The net result is that the binding of GroES and ATP to GroEL which has a substrate bound in its denatured form is to release the denatured substrate either into the cavity or into solution where it can refold.

Minichaperones have been described in detail elsewhere (see International patent application WO99/05163, the disclosure of which in incorporated herein by reference). Minichaperone polypeptides possess chaperoning activity when in monomeric form and do not require energy in the form of ATP. Defined fragments of the apical domain of GroEL of approximately 143-186 amino acid residues in length have molecular chaperone activity towards proteins either in solution under monomeric conditions or when monodisperse and attached to a support.

### SUMMARY OF THE INVENTION

The activity of minichaperones, although sufficient for many purposes, is inferior to that of intact GroEL. It is postulated that this could be due to the inability of minichaperones to oligomerise. There is thus a widespread requirement for a system which would allow the oligomerisation of polypeptides to form functional protein oligomers which have activities which surpass those of recombinant monomeric polypeptides.

According to the present invention, there is provided a polypeptide monomer capable of oligomerisation, said monomer comprising an heterologous amino acid sequence inserted into the sequence of a subunit of an oligomerisable protein scaffold.

It has been observed that the oligomerisation of heterologous polypeptides allows their spatial juxtaposition, which may potentiate their activity. Where the activity is or involves binding, oligomerisation significantly increases the avidity of binding over that which is observed with monomers. Moreover, if the oligomer is heterogeneous, oligomeric constructs according to the invention permit the juxtaposition of a plurality of biological activities which can be brought to bear on a single molecule contemporaneously.

A protein scaffold is a protein, or part thereof, whose function is to determine the structure of the protein itself, or of a group of associated proteins or other molecules. Scaffolds therefore have a defined three-dimensional structure when assembled, and have the capacity to support molecules or polypeptide domains in or on the said structure. Advantageously, a scaffold has the ability to assume a variety of viable geometries, in relation to the three-dimensional structure of the scaffold and/or the insertion site of the heterologous polypeptides.

Preferably, the scaffold according to the invention is a chaperonin cpn10/Hsp10 scaffold. Cpn10 is a widespread component of the cpn60/cpn10 chaperonin system. Examples of cpn10 include bacterial GroES and bacteriophage T4 Gp31. Further members of the cpn10 family will be known to those skilled in the art.

The invention moreover comprises the use of derivatives of naturally-occurring scaffolds. Derivatives of scaffolds (including scaffolds of the cpn10 and 60 families) comprise mutants thereof, which may contain amino acid deletions, additions or substitutions (especially replacement of Cys residues in Gp31), hybrids formed by fusion of different members of the Cpn10 or 60 families and/or circular permutated protein scaffolds, subject to the maintenance of the “oligomerisation” property described herein.

Protein scaffold subunits assemble to form a protein scaffold. In the context of the present invention, the scaffold may have any shape and may comprise any number of subunits. Preferably, the scaffold comprises between 2 and 20 subunits, advantageously between 5 and 15 subunits, and ideally about 10 subunits. The scaffold of cpn10 family members comprises seven subunits, in the shape of a seven-membered ring or annulus. Advantageously, therefore, the scaffold is a seven-membered ring.

Advantageously, the heterologous amino acid sequence, which may be a single residue such as cysteine which allows for the linkage of further groups or molecules to the scaffold, is inserted into the sequence of the oligomerisable protein scaffold subunit such that both the N and C termini of the polypeptide monomer are formed by the sequence of the oligomerisable protein scaffold subunit. Thus, the heterologous polypeptide is included with the sequence of the scaffold subunit, for example by replacing one or more amino acids thereof.

It is known that cpn10 subunits possess a “mobile loop” within their structure. The mobile loop is positioned between amino acids 15 and 34, preferably between amino acids 16 to 33, of the sequence of E. coli GroES, and equivalent positions on other members of the cpn10 family. The mobile loop of T4 Gp31 is located between residues 22 to 45, advantageously 23 to 44. Advantageously, the heterologous polypeptide is inserted by replacing all or part of the mobile loop of a cpn10 family polypeptide.

Where the protein scaffold subunit is a cpn10 family polypeptide, the heterologous sequence may moreover be incorporated at the N or C terminus thereof, or in positions which are equivalent to the roof β hairpin of cpn10 family peptides. This position is located between positions 54 and 67, advantageously 55 to 66, and preferably 59 and 61 of bacteriophage T4 Gp31, or between positions 43 to 63, preferably 44 to 62, advantageously 50 to 53 of E. coli GroES.

Advantageously, the polypeptide may be inserted at an N or C terminus of a scaffold subunit in association with circular permutation of the subunit itself. Circular permutation is described in Graf and Schachman, PNAS(USA) 1996, 93:11591. Essentially, the polypeptide is circularised by fusion of the existing N and C termini, and cleavage of the polypeptide chain elsewhere to create novel N and C termini. In a preferred embodiment of the invention, the heterologous polypeptide may be included at the N and/or C terminus formed after circular permutation. The site of formation of the novel termini may be selected according to the features desired, and may include the mobile loop and/or the roof β hairpin.

Advantageously, heterologous sequences, which may be the same or different, may be inserted at more than one of the positions above-identified in the protein scaffold subunit. Thus, each subunit may comprise two or more heterologous polypeptides, which are displayed on the scaffold when this is assembled.

Heterologous polypeptides may be displayed on a scaffold subunit in free or constrained form, depending on the degree of freedom provided by the site of insertion into the scaffold sequence. For example, varying the length of the sequences flanking the mobile or β hairpin loops in the scaffold will modulate the degree of constraint of any heterologous polypeptide inserted therein.

In a second aspect, the invention relates to a polypeptide oligomer comprising two or more monomers according to the first aspect of the invention. The oligomer may be configured as a heterooligomer, comprising two or more different amino acid sequences inserted into the scaffold, or as a homooligomer, in which the sequences inserted into the scaffold are the same.

If the oligomer according to the invention is a heterooligomer, it may be configured such that the polypeptides juxtaposed thereon have complementary biological activities. For example, two enzymes which act on the same substrate in succession are advantageously displayed on the same scaffold, enabling them to act in concert.

The monomers which constitute the oligomer may be covalently crosslinked to each other. Cross linking may be performed by recombinant approaches, such that the monomers are expressed ab initio as an oligomer; alternatively, cross-linking may be performed at Cys residues in the scaffold. For example, unique Cys residues inserted between positions 50 and 53 of the GroES scaffold, or equivalent positions on other members of the cpn10 family, may be used to cross-link scaffold subunits.

The nature of the heterologous polypeptide inserted into the scaffold subunit in accordance with the present invention may be selected at will. Examples of possible applications of the technology of the invention are set forth below; however, it will be apparent to the person skilled in the art that many different applications of the invention can be envisaged and, with the benefit of the present disclosure, put into practice in a straight forward manner.

Particularly advantageous embodiments of the invention include proteins which display antibodies, particularly fragments thereof such as scFv, natural or camelised V_{H} domains and V_{H} CDR3 fragments; antigens, for example for vaccination; and polypeptides which have a biological activity, such as enzymes.

In a further aspect, the present invention relates to a method for preparing a polypeptide monomer capable of oligomerisation according to the first aspect of the invention, comprising the steps of inserting a nucleic acid sequence encoding a heterologous polypeptide into a nucleic acid sequence encoding a subunit of an oligomerisable protein scaffold, incorporating the resulting nucleic acid into an expression vector, and expressing the nucleic acid to produce the polypeptide monomers.

The invention moreover relates to a method for producing a polypeptide oligomer according to the second aspect of the invention, comprising allowing the polypeptide monomers produced as above to associate into an oligomer. Preferably, the monomers are cross-linked to form the oligomer.

### BRIEF DESCRIPTION OF THE DRAWINGS

FIG. 1. (a) Three-dimensional structure of Gp31 of bacteriophage T4 solved at 2.3 Å. Positions mentioned in the text are indicated (residues numbered as in van der Vies, S., Gatenby, A. & Georgopoulos, C. (1994) Nature 368, 654-656). (b) Three-dimensional structure of minichaperone GroEL(191-376) solved at 1.7 Å. The distance between residues 25 and 43 of Gp31 is around 12 Å; the distance between residues 191 and 376 of GroEL is around 9 Å. Positions mentioned in the text are indicated (residues numbered as in Hemmingsen, S. M., Woolford, C., van, d. V. S., Tilly, K., Dennis, D. T., Georgopoulos, C. P., Hendrix, R. W. & Ellis, R. J. (1988) Nature 333, 330-334.). Secondary structure representations are drawn with MolScript (Kraulis, P. (1991) J. Appl. Crystallogr. 24, 946-950).

FIG. 2. Schematic representation of Gp31 proteins in the vectors used in this study. The presence of the Gp31 mobile loop (residues 23 to 44) and/or minichaperone GroEL (residues 191 to 376) are indicated by boxes. The nucleotide sequence of the Gp31 mobile loop and relevant restriction sites are shown. The names of the corresponding vector are listed in the left margin.

FIG. 3. (a) Molecular weight determination by analytical gel filtration chromatography. Wild-type proteins Gp31 (Mᵣ≈7×12 kDa) and GroEL(191-376) (Mᵣ≈22 kDa) and, Gp31Δloop and Gp31Δ::GroEL(191-376) (MC₇) were run on a Superdex™ 200 (HR 10/30) column (Pharmacia Biotech.) calibrated with molecular weight standards (solid-line and circles). Gp31Δloop and MC₇ eluted at volumes corresponding to molecular weights of ≈145.6 and ≈215 kDa, respectively. (b) Molecular weight determination of MC₇ by equilibrium analytical ultracentrifugation. The apparent molecular weight of MC₇ is ≈215 kDa.

FIG. 4. Characterisation of MC₇ by CD spectroscopy. (a) Far UV-CD spectrum at 25° C. (b) Thermal denaturation followed at 222 nm at a heating rate of 1° C.min⁻¹.

FIG. 5. (a) Binding specificity of MC₇ to GroES determined by ELISA. (b) Inhibition of MC₇, binding to heptameric co-chaperonin GroES by varying concentrations of synthetic peptide corresponding to residues 16 to 32 of GroES mobile loop determined by competition ELISA.

FIG. 6. Binding avidity of MC₇ to anti-GroEL antibodies determined (a) by direct ELISA or (b) by indirect ELISA.

FIG. 7. In vitro refolding of heat- and dithiothreitol-denatured mtMDH. (a) Protection of aggregation at 47° C. followed by light scattering at 550 nm. (b) Time-dependent reactivation of mtMDH at 25° C. (c) Yields of mtMDH reactivation.

FIG. 8 and FIG. 9 show the possible insertion sites for heterologous polypeptide sequences or single amino acids to a scaffold, either bacteriophage T4 Gp31 (FIG. 8) or bacterial GroES (FIG. 9).

FIG. 10 illustrates the potential attachment sites for heterologous polypeptide sequences to a scaffold, in this case bacterial GroES.

FIG. 11 shows a number of applications of scaffolded polypeptides, including oligomerisation of antibody binding domains, optionally including a label such as GFP, and potentially purification and/or cellular targetting tags.

FIG. 12 illustrates further applications for scaffolded polypeptides, including the formation of heterooligomers having a plurality of different functionalities, and the use of a circularly permuted subunit as a two-hybrid system.

### DETAILED DESCRIPTION OF THE INVENTION

Definitions

Oligomerisable scaffold. An oligomerisable scaffold, as referred to herein, is a polypeptide which is capable of oligomerising to form a scaffold and to which a heterologous polypeptide may be fused, preferably covalently, without abolishing the oligomerisation capabilities. Thus, it provides a “scaffold” using which polypeptides may be arranged into multimers in accordance with the present invention. Optionally, parts of the wild-type polypeptide from which the scaffold is derived may be removed, for example by replacement with the heterologous polypeptide which is to be presented on the scaffold.

Monomer. Monomers according to the present invention are polypeptides which possess the potential to oligomerise. This is brought about by the incorporation, in the polypeptide, of an oligomerisable scaffold subunit which will oligomerise with further scaffold subunits if combined therewith.

Oligomer. As used herein, “oligomer” is synonymous with “polymer” or “multimer” and is used to indicate that the object in question is not monomeric. Thus, oligomeric polypeptides according to the invention comprise at least two monomeric units joined together covalently or non-covalently. The number of monomeric units employed will depend on the intended use of the oligomer, and may be between 2 and 20 or more. Advantageously, it is between 5 and 10, and preferably about 7.

Polypeptide. As used herein, a polypeptide is a molecule comprising at least one peptide bond linking two amino acids. This term is synonymous with “protein” and “peptide”, both of which are used in the art to describe such molecules. A polypeptide may comprise other, non-amino acid components. A heterologous polypeptide is a polypeptide which is heterologous to the protein scaffold used in the invention. In other words, it is not part of the same molecule in nature. It may be derived from the same organism. Examples of polypeptides include those used for medical or biotechnological use, such as interleukins, interferons, antibodies and their fragments, insulin, transforming growth factor, antigens, immunogens and many toxins and proteases.

### DESCRIPTION OF PREFERRED EMBODIMENTS

Scaffold Proteins

In a preferred embodiment, the scaffold polypeptide is based on members of the cpn10/Hsp10 family, such as GroES or an analogue thereof. A highly preferred analogue is the T4 polypeptide Gp31. GroES analogues, including Gp31, possess a mobile loop (Hunt, J. F., et al., (1997) Cell 90, 361-371; Landry, S. J., et al, (1996) Proc. Natl. Acad. Sci. U.S.A. 93, 11622-11627) which may be inserted into, or replaced, in order to fuse the heterologous polypeptide to the scaffold.

Cpn10 homologues are widespread throughout animals, plants and bacteria. For example, a search of GenBank indicates that cpn10 homologues are known in the following species:

Actinobacillus actinomycetemcomitans; Actinobacillus pleuropneumoniae; Aeromonas salmonicida; Agrobacterium tumefaciens; Allochromatium vinosum; Amoeba proteus symbiotic bacterium; Aquifex aeolicus; Arabidopsis thaliana; Bacillus sp; Bacillus stearothermophilus; Bacillus subtilis; Bartonella henselae; Bordetella pertussis; Borrelia burgdorferi; Brucella abortus; Buchnera aphidicola; Burkholderia cepacia; Burkholderia vietnamiensis; Campylobacter jejuni; Caulobacter crescentus; Chlamydia muridarum; Chlamydia trachomatis; ChIamydophila pneumoniae; Clostridium acetobutylicum; Clostridium perfringens; Clostridium thermocellum; coliphage T; Cowdria ruminantium; Cyanelle Cyanophora paradoxa; Ehrlichia canis; Ehrlichia chaffeensis; Ehrlichia equi; Ehrlichia phagocytophila; Ehrlichia risticii; Ehrlichia sennetsu; Ehrlichia sp ‘HGE agent’; Enterobacter aerogenes; Enterobacter agglomerans; Enterobacter amnigenus; Enterobacter asburiae; Enterobacter gergoviae; Enterobacter intermedius; Erwinia aphidicola; Erwinia carotovora; Erwinia herbicola; Escherichia coli; Francisella tularensis; Glycine max; Haemophilus ducreyi; Haemophilus influenzae Rd; Helicobacter pylori ; Holospora obtusa; Homo sapiens; Klebsiella ornithinolytica; Klebsiella oxytoca; Klebsiella planticola; Klebsiella pneumoniae; Lactobacillus helveticus; Lactobacillus zeae; Lactococcus lactis; Lawsonia intracellularis; Leptospira interrogans; Methylovorus sp strain SS; Mycobacterium avium; Mycobacterium avium subsp avium; Mycobacterium avium subsp paratuberculosis; Mycobacterium leprae; Mycobacterium tuberculosis; Mycoplasma genitalium; Mycoplasma pneumoniae; Myzus persicae primary endosymbiont; Neisseria gonorrhoeae; Oscillatoria sp NKBG; Pantoea ananas; Pasteurella multocida; Porphyromonas gingivalis; Pseudomonas aeruginosa; Pseudomonas aeruginosa; Pseudomonas putida; Rattus norvegicus; Rattus norvegicus; Rhizobium leguminosarum; Rhodobacter capsulatus; Rhodobacter sphaeroides; Rhodothermus marinus; Rickettsia prowazekii; Rickettsia rickettsii; Saccharomyces cerevisiae; Serratia ficaria; Serratia marcescens; Serratia rubidaea; Sinorhizobium meliloti; Sitophilus oryzae principal endosymbiont; Stenotrophomonas maltophilia; Streptococcus pneumoniae; Streptomyces albus; Streptomyces coelicolor; Streptomyces coelicolor; Streptomyces lividans; Synechococcus sp; Synechococcus vulcanus; Synechocystis sp; Thermoanaerobacter brockii; Thermotoga maritima; Thermus aquaticus; Treponema pallidum; Wolbachia sp; Zymomonas mobilis.

An advantage of cpn10 family subunits is that they possess a mobile loop, responsible for the protein folding activity of the natural chaperonin, which may be removed without affecting the scaffold.

Cpn10 with a deleted mobile loop possesses no biological activity, making it an advantageously inert scaffold, thus minimising any potentially deleterious effects. Insertion of an appropriate biologically active polypeptide can confer a biological activity on the novel polypeptide thus generated. Indeed, the biological activity of the inserted polypeptide may be improved by incorporation of the biologically active polypeptide into the scaffold.

Alternative sites for peptide insertion are possible. An advantageous option is in the position equivalent to the roof beta hairpin in GroES. This involves replacement of Glu-60 in Gp31 by the desired peptide. The amino acid sequence is Pro(59)-Glu(60)-Gly(61). This is conveniently converted to a Smal site at the DNA level (CCC:GGG) encoding Pro-Gly, leaving a blunt-ended restriction site for peptide insertion as a DNA fragment.

Similarly, an insertion may be made at between positions 50 and 53 of the GroES sequence, and at equivalent positions in other cpn10 family members. Alternatively, inverse PCR may be used, to display the peptide on the opposite side of the scaffold.

Members of the cpn60/Hsp60 family of chaperonin molecules may also be used as scaffolds. For example, the tetradecameric bacterial chaperonin GroEL may be used. Advantageously, heterologous polypeptides would be inserted between positions 191 and 376, in particular between positions 197 and 333 (represented by SacII engineered and unique Cla I sites) to maintain intact the hinge region between the equatorial and the apical domains in order to impart mobility to the inserted polypeptide. The choice of scaffold may depend upon the intended application of the oligomer: for example, if the oligomer is intended for vaccination purposes (see below), the use of an immunogenic scaffold, such as that derived from Mycobacterium tuberculosis, is highly advantageous and confers an adjuvant effect.

Mutants of cpn60 molecules may also be used. For example, the single ring mutant of GroEL (GroELSR1) contains four point mutations which effect the major attachment between the two rings of GroEL (R452E, E461A, S463A and V464A) and is functionally inactive in vitro because it is release to bind GroES. GroELSR2 has an additional mutation at Glu191-Gly, which restores activity by reducing the affinity for GroES. Both of these mutants for ring structures and would be suitable for use as scaffolds.

Polypeptides

Any polypeptide or amino acid, such as cysteine, may be incorporated into the structure of the monomers or oligomers as described above. The following classes of polypeptide are preferred, but the invention is not limited thereto.

Immunogens

Immunogenic peptides, capable of raising an immune response when exposed to the immune system of an organism, are preferred polypeptides for insertion into monomers and oligomers according to the invention. This aspect of the invention has many applications, not only in vaccination but also in research. For example, the generation of human gene sequence data by the human genome project has made the generation of antisera reactive to new polypeptides a pressing requirement. The same requirement applies to prokaryotic, such as bacterial, and other eukaryotic, including fungal, gene products. Incorporation of more than one polypeptide immunogen into a scaffold increases the efficiency of the immunogens, due to increased avidity for immunoglobulin molecules.

The present invention has many advantages in the generation of an immune response. For example, the use of oligomers can permit the presentation of a number of antigens, simultaneously, to the immune system. This allows the preparation of polyvalent vaccines, capable of raising an immune response to more than one epitope, which may be present on a single organism or a number of different organisms. Thus, vaccines according to the invention may be used for simultaneous vaccination against more than one disease, or to target simultaneously a plurality of epitopes on a given pathogen. A preferred group of antigenic polypeptides is the V3 loops of various HIV subgroups, which can be inununised against simultaneously by the method of the present invention.

Display of the V3 loop of the envelope glycoprotein gp120 of HIV-1 (and -2) on a polypeptide scaffold is highly advantageous:

1. The production of a series of variant loops allows both sensitive detection of anti-HIV antibodies and simultaneous typing of the infecting subgroup of HIV on an array of loops.

2. As an antigen for eliciting polyclonal or monoclonal antibodies, these scaffolded loops provide very specific epitopes for immunisation and vaccination.

3. The scaffolded loops can be developed further to provide a screening assay of very high throughput to detect which are potential antiviral agents.

The V3 loop of HIV-1 gp120 is the major (but not exclusive) determinant of viral tropism. A substantial body of literature demonstrates that initial binding of CD4 (the primary HIV receptor) to gp120 alters the conformation of the latter, exposing the V3 loop which binds then to one of a number of chemokine receptors on the same cell surface. The chemokine receptor (sometimes called the co-receptor) is usually CCR5 on macrophages and CXCR4 on T-cells, the two most important cell types infected by HIV. Dual tropic strains of HIV exist which can use either co-receptor, and consequently will infect both cell types.

Importantly, while V3 loops are highly variable (entire sections of the Los Alamos HIV database are devoted to recording the variability; see http://hiv-web.lanl.gov) the co-receptors, being host-encoded are not. Compounds which bind tightly to the host's chemokine receptors should therefore be capable of foiling viral entry. In fact the natural ligands for these receptors (RANTES, MIP-1alpha and MIP-1beta for CCR5; SDF or Stem-cell derived factor for CXCR4) do just that.

Scaffolded V3 loops with, for example Green Fluorescent Protein (GFP) from the jellyfish Aequorea Victoria, on the opposite side of the scaffold, act as surrogate labels; compounds may be screened in large numbers for their ability to displace binding of the scaffolded loops to the receptor (see J. Virol 1997 Volume 71, pages 6296-6304 where radio-labelled chemokines were used in such a displacement assay). Labelled chemokines provide useful controls for the specificity of the assay: they should displace the scaffold from the appropriate receptor.

Structure/function studies can be carried out by mutagenesis of the loop (see, for example, EMBO Journal, 1997 Volume 16 pages 2599-2609).

Furthermore, the display of the CDR2-like loop of the CD4 receptor on the scaffold increases the affinity for gp120 and consequently inhibits infection of CD4+ T-cells by HIV-1 viruses.

Moreover, the invention may be exploited by incorporating an adjuvant on the scaffold, together with the immunogen. Suitable adjuvants are, for example, bacterial toxins and cytokines, such as interleukins. The potency of the immunogen is thereby increased, allowing more efficient raising of antisera and more efficient immunisation.

Preferably, in the context of immunisations, a bacterial or bacteriophage scaffold is used. Such scaffolds are unlikely to encounter endogenous host antibodies upon administration, since naturally-occurring antibodies to these molecules are rare.

The invention may be applied to the detection or the neutralisation of antibodies in vivo or in vitro. For example, in vitro, polyvalent or monovalent antigen-bearing scaffolds may be used to select antibody molecules derived from phage display experiments. Moreover, in vivo, antigen-bearing scaffolds according to the invention may be used to neutralise antoantibodies in autoimmune disease, or to detect antibodies which may be indicative of pathological conditions, such as in HIV testing or other diagnostic applications.

Polyvalent Polypeptide Antigens and Vaccines

A major application of the Scaffold technology is the use of the assembled peptides or polypeptides as antigens. The oligomerisation improves both detection of antibodies against, and the induction of antibodies to, such antigens. Some of these antigens may be of prophylactic value; they might be useful for vaccination. The method allows rapid progress from nucleotide sequences to the production of recombinant antigens in a polyvalent form. Predicted open reading frames (ORFs) can be used to design oligonucleotide sequences encoding the predicted protein sequence. Cloning of these oligonucleotides into the Cpn10 scaffold vectors allows a very rapid production of antigens, without, for example the need for isolating cDNAs and expressing them in heterologous systems such as Escherichia coli. The avidity effect of the heptameric structure of MC₇ (a chimeric GroEL minichaperone displayed on T4 Gp31 scaffold as described herein) was confirmed by analysing the binding of antibodies specific to GroEL; comparable detection levels were observed for GroEL and MC₇ at the different concentrations of antibodies used. In addition, using affinity panning of immobilised MC₇ for a large library of bacteriophages (“phage display”) that display single-chain Fv (scFv) antibodies fragments, we selected recombinant monoclonal scFvs that recognised only and specifically GroEL(191-376), and not the scaffold, Gp31Δloop.

An attractive feature of Scaffold is that it is a bacteriophage product: for this reason, naturally occurring antibodies to it are rare. This enhances the use of Scaffold fusions as vaccine agents. T4 Gp31 with a deleted loop has no biological activity (except as a dominant-negative or intracellular vaccine against T4 bacteriophage) thus minimising deleterious effects on the host. However, insertion of appropriate sequences encoding polypeptides can confer biological activity on the novel proteins. Indeed, the biological activity may be improved by insertion into the Scaffold.

Antibodies

The affinity of antibodies or antibody fragments for antigens may be increased by oligomerisation according to the present invention. Antibody fragments may be fragments such as Fv, Fab and F(ab′)₂ fragments or any derivatives thereof, such as a single chain Fv fragments. The antibodies or antibody fragments may be non-recombinant, recombinant or humanised. The antibody may be of any immunoglobulin isotype, e.g., IgG, IgM, and so forth.

In a preferred aspect, the antibody fragments may be camelised V_{H} domains. It is known that the main intermolecular interactions between antibodies and their cognate antigens are mediated through V_{H} CDR3. However, V_{H}-only antibodies, such as those derived from camel or llama (natrually V_{H}-only single chain antibodies), have only low affinity for cognate antigen.

The present invention provides for the oligomerisation of V_{H} domains, or V_{H} CDR3 domains, to produce a high-affinity antibody. Two or more domains may be included in an oligomer according to the invention; in an oligomer based on a cpn10 scaffold, up to 7 domains may be included, forming a hetpameric antibody molecule (heptabody). Advantageously, the antibody domains are arranged in a seven-membered ring formation, based on the cpn10 scaffold.

Receptor Ligands

Many ligand-receptor pairs depend on dimerisation for activation of the receptor. Examples include the insulin and erythropoietin receptors. The function of the ligand is to dimerise the receptor, which leads to autophosphorylation and hence activation of the receptor. Whilst some ligands, such as substance P, are short polypeptides, others (including kinase and phosphatase substrates) are complex molecules which possess binding loops projecting from the surface thereof. Short peptides or loops may be incorporated into oligomers according to the present invention to form a polyvalent receptor ligand or kinase/phosphatase substrate, useful for activating or inhibiting receptors and/or kinases at very low concentrations.

Variation may be introduced into the heterologous polypeptides inserted into the scaffold in order to map the specificity of receptors or kinases/phosphatases for their ligands/substrates. Variants may be produced of the same loop, or a set of standard different loops may be devised, in order to assess rapidly the specificity of a novel kinase/phosphatase. Variants may be produced by randomisation of sequences according to known techniques, such as PCR. They may be subjected to selection by a screening protocol, such as phage display, before incorporation into protein scaffolds in accordance with the invention.

Enzymes

Numerous biological reactions involve the sequential, and/or synergistic, action of a plurality of protein activities. Such protein activities may be incorporated into a single molecule in accordance with the present invention. Preferably, therefore, the monomers which are used to compose the oligomer according to the invention incorporate amino acid sequences which encode distinct biological activities. The activities are advantageously complementary, such that they are required sequentially in a biological reaction, or act synergistically. The invention therefore provides plurifuntional macromolecular structures.

Polyvalent Receptor Ligands

Many cell surface receptors are activated by dimerisation. Well known examples are those for insulin and erythropoietin. The function of the ligand is to bind simultaneously to two receptors, thus dimerising and activating them. In the examples cited, receptor autophosphorylation occurs. This activates the receptor, which has a tyrosine kinase domain in its intracellular portion. The kinase is inactive when the receptor is monomeric, but is activated on dimerisation. This triggers a cascade of intracellular events, collectively referred to as signal transduction.

Some ligands whose receptors are activated by dimerisation (or oligomerisation) are large proteins (insulin is 51 kDa). Smaller molecules which can mimic the natural ligands for receptors are useful for research purposes (for example to understand the specificity of ligand receptor binding). Other receptor ligands are rather short peptides (e.g. substance P); oligomerisation of these peptide sequences on a scaffold enables such ligands to be artificially oligomerised, thus activating or inhibiting their receptors at very low concentrations.

Variation of the sequence, in a constrained conformation, provides insight into the structural features of the ligand required for binding and for activation. With larger ligands, e.g. erythropoietin, small fragments of the ligand can be presented in a constrained conformation allowing “mapping” of residues essential for ligand binding. The oligomerisation allows functional assay of the constrained peptides by receptor autophosphorylation, for example.

Receptor dimerisation or oligomerisation mediated by scaffold constructs can also be used to inhibit HIV infection, even though G-protein coupled receptors are not thought to require dimerisation for activity. A recent paper (ref: A. J. Vila-Coro, M. Mellado, A. Martin de Ana, P. Lucas, G. del Real, C. Martinez-A.,and J. M. Rodriguez-Frade. Proc.Natl.Acad.Sci. USA 2000 Volume 97, pages 3388-3394 entitled “HIV-1 infection through the CCR5 receptor is blocked by receptor dimerization”) shows that an antibody that neither triggers receptor down-regulation nor interferes with the gp120 binding to CCR5 blocks HIV-1 replication in both in vitro assays and in vivo. This anti-CCR5 mAb efficiently prevents HIV-1 infection by inducing receptor dimerization. Note that chemokine receptor dimerization was also induced by chemokines and was required for their anti-HIV-1 activity.

Phage Display

Phage display technology has proved to be enormously useful in biological research. It enables ligands to be selected from large libraries of molecules. Scaffold technology also harnesses the power of this technique, but with some powerful advantages over normal applications. Cpn10 molecules can be displayed as monomers on fd bacteriophages, just as single-chain Fv molecules are. Libraries of insertions (in place of the highly mobile loop) are constructed by standard methods, and the resulting libraries screened for ligands of interest. It is important to note that this is an affinity based selection. After characterisation, the ligands selected for affinity, can be oligomerised, and thus take advantage of avidity. When the target for the ligand is oligomeric, very tight binding will result. Furthermore, ligands selected as monomers, will be able to cross-link or oligomerise their binding partners. An obvious application of this effect is in triggering receptor activation; see above.

Kinase Substrates

Protein kinase cascades or pathways are involved in a very wide range of signal transduction pathways of biological interest. The substrate sites for many kinases are known to form loops projecting from the surface of the protein substrate. Peptides constrained on Scaffold are useful mimics of such molecules and particularly in delineating the substrate specificity of (e.g recombinant ) kinases, either as a library of variants of the same loop, or as a set of standard different loops to assay quickly the substrate specificity of novel kinases. Scaffold greatly simplifies the construction of such libraries; all that is required is the cloning by standard methods of double-stranded oligonucleotides encoding the desired protein sequence into a restriction site (for example the BamH I site of Gp31Δloop).

This obviates the requirement for purifying multiple different standard substrate proteins, and greatly simplifies the determination of the substrate specificity of both known and novel kinases. It is particularly advantageous to create arrays, or “protein chips” containing (potentially) very large numbers of kinase substrate loops to assayed in parallel.

Presentation of the loops on bacteriophages (see above) allows large numbers of variant sequences to be assayed simultaneously. An example of the use of such libraries is in screening for protein kinase substrate specificity. The library is first phosphorylated with the kinase of interest in the presence of gamma-thio- ATP which will phosphorylate only a subset of phage in any pool. These modified targets can then be selectively biotinylated (see BioTechniques 2000 for details of the method). Streptavidin is then used to purify the phage of interest. Repetition of this selection allows the sequence being phosphorylated to be determined, after a number of rounds.

Protein Chips

Currently, DNA microarrays, whether of oligonucleotides, PCR products or cloned DNAs, are major tools enabling rapid development in the highly parallel analysis of gene expression. Clearly, in many situations, it would be far preferable to monitor gene expression directly, that is, by assaying protein expression levels rather than mRNA levels. The latter are but an indirect measure of gene activity which rely on the hybridisation of labelled cDNA and can be very misleading because they is often a poor correlation between the abundance of a particular mRNA and the frequency at which it is translated into proteins. In addition, mRNA analysis can not possibly determine whether the encoded protein, even if translated, is active. This may depend on post-translational modification.

Scaffold technology enables thousands of protein-protein interactions to be monitored in parallel. An array of distinct scaffolded protein aptamers [see Norman, T. C. et al. (1999). Genetic selection of peptide inhibitors of biological pathways. Science 285, 591-595] each specific for a specific protein, or a post-translationally modified protein, can serve as a matrix for binding and quantitating labelled proteins, however heterologous the initial mixture. An attractive feature of the Scaffold system is that the individual arrayed, oligomers of aptamers can be oriented, at the molecular level on the slide or matrix, by incorporating specific sequences, for example poly-L-Lysine in the scaffold on the opposite side to the aptamers. This ensures that most of the molecules “stand” on their poly-L-Lysine “legs” (and thus stick to DNA glass slides) while the aptamer sequence projects in a favourable orientation for binding its ligand.

Carriers for DNA Vaccines

Vaccination using DNA represents a major advance in immunisation methods and promises enormous benefits in preventative medicine. DNA can be administered for this purpose “naked”, but in this form it is susceptible to degradation by nucleases and is relatively inefficiently taken up by cells. It is preferably administered coated with proteins to minimise degradation and to enhance cellular uptake. In addition, the protective protein may have adjuvant properties. This applies especially to Hsp60, and fragments thereof, which are known to have strong immunostimulatory properties.

To ensure efficient coating of the DNA in order to protect it from degradation, any of a large number of oligomerised peptides can be used. These preferably contain several basic residues, for example lysine and arginine, to ensure efficient and avid binding to the DNA. Histones, or fragments thereof, provide examples. Immunogenicity can be minimised by using the sequences of host proteins as a scaffold (e.g. Hsp10 and Hsp60) and as the insertion (e.g. histones). A further advantage of these proteins is that they are highly conserved in sequence, minimising the number of modification that have to be made for different species.

The target cells to which DNA vaccines should ideally be delivered are those responsible for antigen presentation. These are highly specialised cells with a recognised ability to take up particulate material. It is far from clear that current DNA vaccination regimes are actually delivering DNA directly to these cells. Instead it is more likely that non-immune cells are being transfected and that these are presenting the antigens derived from transcription and translation of the encoded polypeptides. This is a less potent means of generating an immune response than direct delivery to professional antigen presenting cells.

Scaffold as a Pluri-functional Macromolecular Structure

Numerous biological reactions involve, sequentially or synergistically, different proteins with different activities. Different polypeptides, for example enzymes (particularly when these are involved in the same metabolic pathway, or when they are being added as a unit for metabolic engineering) with different activities could be displayed on a scaffold, or on a multimeric structure composed of different subunits, to generate a pluri-functional macromolecular structure.

In a preferred embodiment, the heterologous amino acid sequences are antibiotics. This provides an antibiotic molecule with any desired spectrum of activity.

Configurations of Oligomers According to the Invention

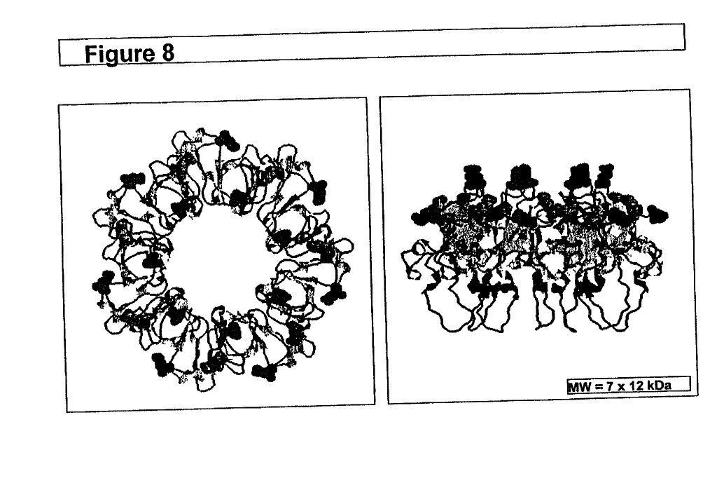

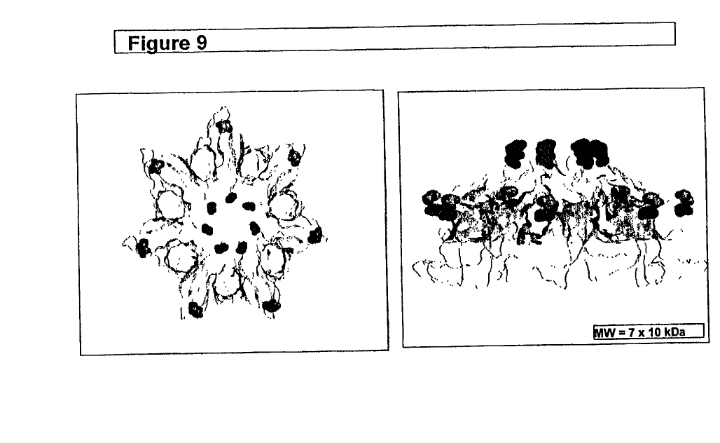

FIGS. 8-12 show various topologies and applications for scaffolded polypeptides in accordance with the present invention. In FIGS. 8 and 9, the possible insertion sites for heterologous polypeptides are shown. Insertion of polypeptides may be performed by any suitable technique, including those set forth by Doi and Yanagawa (FEBS Letters (1999) 457:1-4). As set forth therein, insertion of polypeptides may be combined with randomisation to produce libraries of polypeptide repertoires, suitable for display and selection.

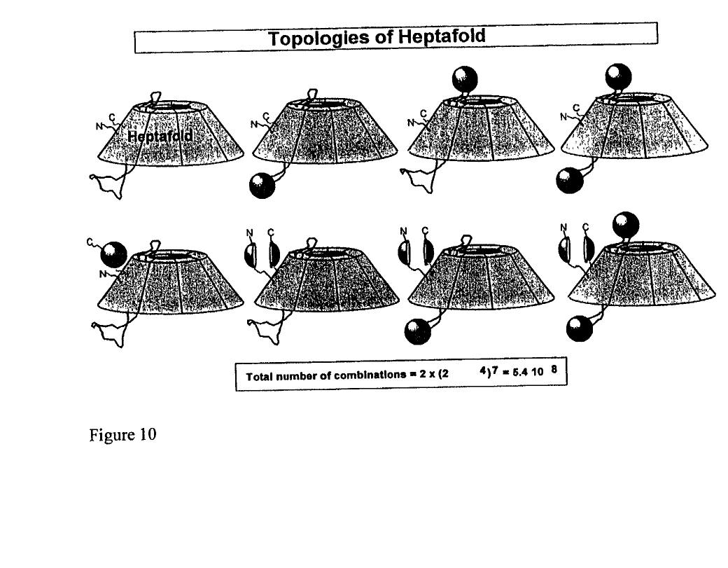

FIG. 10 illustrates the potential attachment sites for heterologous peptide sequences to a circular scaffold, in this case bacterial GroES. Reading from left to right, the figure shows: no attachment, attachment to the mobile loop, attachment to the roof β0 hairpin, attachment at both the mobile loop and the roof β hairpin, attachment at the C terminus, attachment at both N and C termini, attachment at both N and C termini and the mobile loop, and attachment at both N and C termini, the roof β hairpin and the mobile loop. As will be apparent, further configurations are possible, and can be combined in any way in the heptamer, leading to a total of 5.4×10⁸ possible configurations.

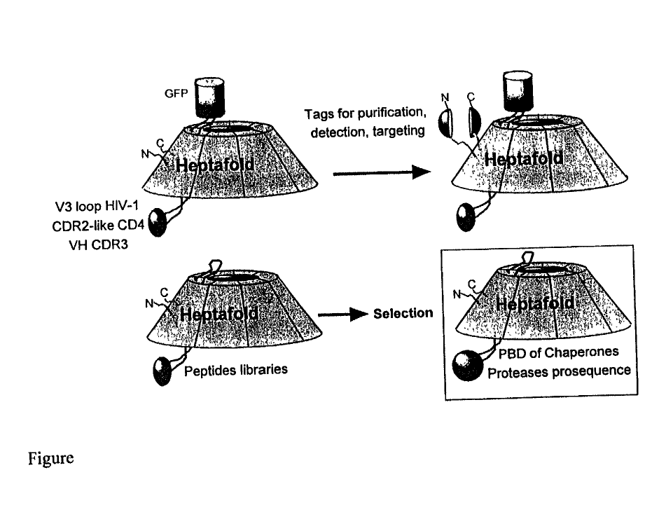

FIG. 11 shows a number of applications of scaffolded polypeptides, including oligomerisation of antibody binding domains, optionally including a label such as GFP, and potentially purification and/or cellular targetting tags. Moreover, the scaffold can be used as a basis for peptide libraries, which may be selected to identify a desired activity.

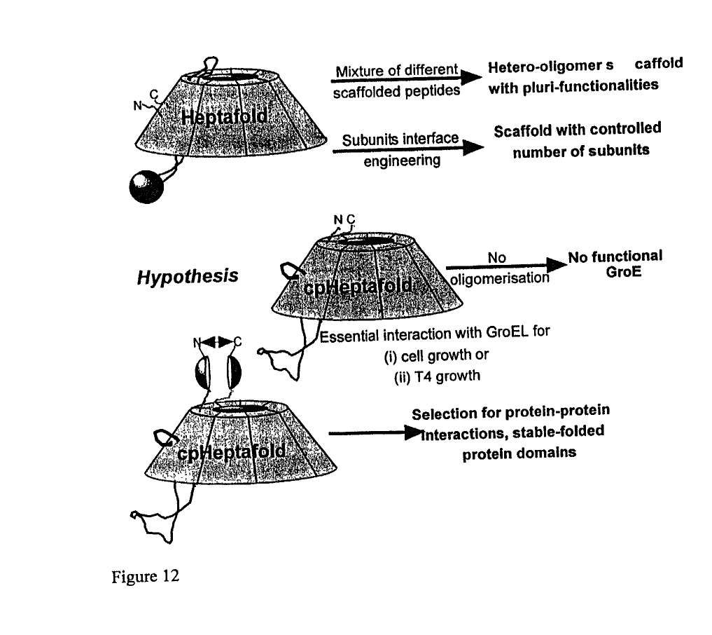

FIG. 12 illustrates further applications for scaffolded polypeptides, including the formation of heterooligomers having a plurality of different functionalities, and the use of a circularly permuted subunit which is incapable of assembly into a ring due to N and C terminus separation, to screen for possible binding pairs; polypeptides placed at the N and C termini will restore ring-forming ability if they bind, and thus restore the function of a cpn10 chaperonin.

Recombinant DNA Techniques

The present invention advantageously makes use of recombinant DNA technology in order to construct polypeptide monomers and oligomers. Advantageously, polypeptide monomers or oligomers may be expressed from nucleic acid sequences which encode them.

As used herein, vector (or plasmid) refers to discrete elements that are used to introduce heterologous DNA into cells for either expression or replication thereof Selection and use of such vehicles are well within the skill of the artisan. Many vectors are available, and selection of appropriate vector will depend on the intended use of the vector, i.e. whether it is to be used for DNA amplification or for DNA expression, the size of the DNA to be inserted into the vector, and the host cell to be transformed with the vector. Each vector contains various components depending on its function (amplification of DNA or expression of DNA) and the host cell for which it is compatible. The vector components generally include, but are not limited to, one or more of the following: an origin of replication, one or more marker genes, an enhancer element, a promoter, a transcription termination sequence and a signal sequence.

Both expression and cloning vectors generally contain nucleic acid sequence that enable the vector to replicate in one or more selected host cells. Typically in cloning vectors, this sequence is one that enables the vector to replicate independently of the host chromosomal DNA, and includes origins of replication or autonomously replicating sequences. Such sequences are well known for a variety of bacteria, yeast and viruses. The origin of replication from the plasmid pBR322 is suitable for most Gram-negative bacteria, the 2m plasmid origin is suitable for yeast, and various viral origins (e.g. SV 40, polyoma, adenovirus) are useful for cloning vectors in mammalian cells. Generally, the origin of replication component is not needed for mammalian expression vectors unless these are used in mammalian cells competent for high level DNA replication, such as COS cells.

Most expression vectors are shuttle vectors, i.e. they are capable of replication in at least one class of organisms but can be transfected into another class of organisms for expression. For example, a vector is cloned in E. coli and then the same vector is transfected into yeast or mammalian cells even though it is not capable of replicating independently of the host cell chromosome. DNA can be amplified by PCR and be directly transfected into the host cells without any replication component.

Advantageously, an expression and cloning vector may contain a selection gene also referred to as selectable marker. This gene encodes a protein necessary for the survival or growth of transformed host cells grown in a selective culture medium. Host cells not transformed with the vector containing the selection gene will not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available from complex media.

As to a selective gene marker appropriate for yeast, any marker gene can be used which facilitates the selection for transformants due to the phenotypic expression of the marker gene. Suitable markers for yeast are, for example, those conferring resistance to antibiotics G418, hygromycin or bleomycin, or provide for prototrophy in an auxotrophic yeast mutant, for example the URA3, LEU2, LYS2, TRP1, or HIS3 gene.

Since the replication of vectors is conveniently done in E. coli, an E. coli genetic marker and an E. coli origin of replication are advantageously included. These can be obtained from E. coli plasmids, such as pBR322, Bluescript© vector or a pUC plasmid, e.g. pUC18 or pUC19, which contain both E. coli replication origin and E. coli genetic marker conferring resistance to antibiotics, such as ampicillin.

Suitable selectable markers for mammalian cells are those that enable the identification of cells which have been transformed, such as dihydrofolate reductase (DHFR, methotrexate resistance), thymidine kinase, or genes conferring resistance to G418 or hygromycin. The mammalian cell transformants are placed under selection pressure which only those transformants which have taken up and are expressing the marker are uniquely adapted to survive. In the case of a DHFR or glutamine synthase (GS) marker, selection pressure can be imposed by culturing the transformants under conditions in which the pressure is progressively increased, thereby leading to amplification (at its chromosomal integration site) of both the selection gene and the linked DNA that encodes the polypeptide according to the invention. Amplification is the process by which genes in greater demand for the production of a protein critical for growth, together with closely associated genes which may encode a desired protein, are reiterated in tandem within the chromosomes of recombinant cells. Increased quantities of desired protein are usually synthesised from thus amplified DNA.

Expression and cloning vectors usually contain a promoter that is recognised by the host organism and is operably linked to the heterologous nucleic acid coding sequence. Such a promoter may be inducible or constitutive. The promoters are operably linked to the coding sequence by inserting the isolated promoter sequence into the vector. Many heterologous promoters may be used to direct amplification and/or expression of the coding sequence. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Promoters suitable for use with prokaryotic hosts include, for example, the β-lactamase and lactose promoter systems, alkaline phosphatase, the tryptophan (trp) promoter system and hybrid promoters such as the tac promoter. Their nucleotide sequences have been published, thereby enabling the skilled worker operably to ligate them to the coding sequence, using linkers or adapters to supply any required restriction sites. Promoters for use in bacterial systems will also generally contain a Shine-Delgarno sequence operably linked to the coding sequence.

Preferred expression vectors are bacterial expression vectors which comprise a promoter of a bacteriophage such as phagex or T7 which is capable of functioning in the bacteria. In one of the most widely used expression systems, the nucleic acid encoding the fusion protein may be transcribed from the vector by T7 RNA polyrnerase (Studier et al, Methods in Enzymol. 185; 60-89, 1990). In the E. coli BL21(DE3) host strain, used in conjunction with pET vectors, the T7 RNA polymerase is produced from the λ-lysogen DE3 in the host bacterium, and its expression is under the control of the IPTG inducible lac UV5 promoter. This system has been employed successfully for over-production of many proteins. Alternatively the polymerase gene may be introduced on a lambda phage by infection with an int- phage such as the CE6 phage which is commercially available (Novagen, Madison, USA). other vectors include vectors containing the lambda PL promoter such as PLEX (Invitrogen, NL) , vectors containing the trc promoters such as pTrcHisXpressTm (Invitrogen) or pTrc99 (Pharmacia Biotech, SE) , or vectors containing the tac promoter such as pKK223-3(Pharmacia Biotech) or PMAL (new England Biolabs, MA, USA).

Moreover, the coding sequence according to the invention preferably includes a secretion sequence in order to facilitate secretion of the polypeptide from bacterial hosts, such that it will be produced as a soluble native peptide rather than in an inclusion body. The peptide may be recovered from the bacterial periplasmic space, or the culture medium, as appropriate.

Suitable promoting sequences for use with yeast hosts may be regulated or constitutive and are preferably derived from a highly expressed yeast gene, especially a Saccharomyces cerevisiae gene. Thus, the promoter of the TRP1 gene, the ADHI or ADHII gene, the acid phosphatase (PH05) gene, a promoter of the yeast mating pheromone genes coding for the a- or α-factor or a promoter derived from a gene encoding a glycolytic enzyme such as the promoter of the enolase, glyceraldehyde-3-phosphate dehydrogenase (GAP), 3-phospho glycerate kinase (PGK), hexokinase, pyruvate decarboxylase, phosphofructokinase, glucose-6-phosphate isomerase, 3-phosphoglycerate mutase, pyruvate kinase, triose phosphate isomerase, phosphoglucose isomerase or glucokinase genes, the S. cerevisiae GAL 4 gene, the S. pombe nmt 1 gene or a promoter from the TATA binding protein (TBP) gene can be used. Furthermore, it is possible to use hybrid promoters comprising upstream activation sequences (UAS) of one yeast gene and downstream promoter elements including a functional TATA box of another yeast gene, for example a hybrid promoter including the UAS(s) of the yeast PH05 gene and downstream promoter elements including a functional TATA box of the yeast GAP gene (PH05-GAP hybrid promoter). A suitable constitutive PHO5 promoter is e.g. a shortened acid phosphatase PH05 promoter devoid of the upstream regulatory elements (UAS) such as the PH05 (-173) promoter element starting at nucleotide -173 and ending at nucleotide -9 of the PH05 gene.

Transcription from vectors in mammalian hosts may be controlled by promoters derived from the genomes of viruses such as polyoma virus, adenovirus, fowlpox virus, bovine papilloma virus, avian sarcoma virus, cytomegalovirus (CMV), a retrovirus and Simian Virus 40 (SV40), from heterologous mammalian promoters such as the actin promoter or a very strong promoter, e.g. a ribosomal protein promoter, provided such promoters are compatible with the host cell systems.

Transcription of a coding sequence by higher eukaryotes may be increased by inserting an enhancer sequence into the vector. Enhancers are relatively orientation and position independent. Many enhancer sequences are known from mammalian genes (e.g. elastase and globin). However, typically one will employ an enhancer from a eukaryotic cell virus. Examples include the SV40 enhancer on the late side of the replication origin (bp 100-270) and the CMV early promoter enhancer. The enhancer may be spliced into the vector at a position 5′ or 3′ to the coding sequence, but is preferably located at a site 5′ from the promoter.

Advantageously, a eukaryotic expression vector may comprise a locus control region (LCR). LCRs are capable of directing high-level integration site independent expression of transgenes integrated into host cell chromatin, which is of importance especially where the coding sequence is to be expressed in the context of a permanently-transfected eukaryotic cell line in which chromosomal integration of the vector has occurred, in vectors designed for gene therapy applications or in transgenic animals.

Eukaryotic expression vectors will also contain sequences necessary for the termination of transcription and for stabilising the mRNA. Such sequences are commonly available from the 5′ and 3′ untranslated regions of eukaryotic or viral DNAs or cDNAs. These regions contain nucleotide segments transcribed as polyadenylated fragments in the untranslated portion of the mRNA.

An expression vector includes any vector capable of expressing nucleic acids that are operatively linked with regulatory sequences, such as promoter regions, that are capable of expression of such DNAs. Thus, an expression vector refers to a recombinant DNA or RNA construct, such as a plasmid, a phage, recombinant virus or other vector, that upon introduction into an appropriate host cell, results in expression of the cloned DNA. Appropriate expression vectors are well known to those with ordinary skill in the art and include those that are replicable in eukaryotic and/or prokaryotic cells and those that remain episomal or those which integrate into the host cell genome. For example, nucleic acids may be inserted into a vector suitable for expression of cDNAs in mammalian cells, e.g. a CMV enhancer-based vector such as pEVRF (Matthias, et al., (1989) NAR 17, 6418).

Construction of vectors according to the invention employs conventional ligation techniques. Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing expression and function are known to those skilled in the art. Gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe which may be based on a sequence provided herein. Those skilled in the art will readily envisage how these methods may be modified, if desired.

The invention also envisages the administration of polypeptide oligomers according to the invention as compositions, preferably for the treatment of diseases associated with protein misfolding. The active compound may be administered in a convenient manner such as by the oral, intravenous (where water soluble), intramuscular, subcutaneous, intranasal, intradermal or suppository routes or implanting (e.g. using slow release molecules). Depending on the route of administration, the active ingredient may be required to be coated in a material to protect said ingredients from the action of enzymes, acids and other natural conditions which may inactivate said ingredient.

In order to administer the combination by other than parenteral administration, it will be coated by, or administered with, a material to prevent its inactivation. For example, the combination may be administered in an adjuvant, co-administered with enzyme inhibitors or in liposomes. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin.

Liposomes include water-in-oil-in-water CGF emulsions as well as conventional liposomes.

The active compound may also be administered parenterally or intraperitoneally. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene gloycol, and the like), suitable mixtures thereof, and vegetable oils. The proper fluidity can be maintained, for example, by the use of a coating such as lecithin, by the maintenance of the required particle size in the case of dispersion and by the use of superfactants.

The prevention of the action of microorganisms can be brought about by various antibacterial and antifungal agents, for example, parabens, chlorobutanol, phenol, sorbic acid, thirmerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compound in the required amount in the appropriate solvent with various of the other ingredients enumerated above, as required, followed by filtered sterilisation. Generally, dispersions are prepared by incorporating the sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the combination of polypeptides is suitably protected as described above, it may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. The amount of active compound in such therapeutically useful compositions in such that a suitable dosage will be obtained.

The tablets, troches, pills, capsules and the like may also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such as active material for the treatment of disease in living subjects having a diseased condition in which bodily health is impaired.

The principal active ingredients are compounded for convenient and effective administration in effective amounts with a suitable pharmaceutically acceptable carrier in dosage unit form. In the case of compositions containing supplementary active ingredients, the dosages are determined by reference to the usual dose and manner of administration of the said ingredients.

In a further aspect there is provided the combination of the invention as hereinbefore defined for use in the treatment of disease. Consequently there is provided the use of a combination of the invention for the manufacture of a medicament for the treatment of disease associated with aberrant protein/polypeptide structure. The aberrant nature of the protein/polypeptide may be due to misfolding or unfolding which in turn may be due to an anomalous e.g. mutated amino acid sequence. The protein/polypeptide may be destabilised or deposited as plaques e.g. as in Alzheimer's disease. The disease might be caused by a prion. A polypeptide-based medicament of the invention would act to renature or resolubilise aberrant, defective or deposited proteins.

The invention is further described below, for the purposes of illustration only, in the following examples.

### EXAMPLES

1. General Experimental Procedures

Bacterial and bacteriophage strains. The E. coli strains used in this study were: C41(DE3), a mutant of BL21(DE3) capable of expressing toxic genes (Miroux, B. & Walker, J. E. (1996) J. Mol. Biol. 260, 289-298); SV2 (B178groEL44), SV3 (B178groEL59) and SV6 (B178groEL673): isogenic strains carrying temperature-sensitive alleles of groEL; SV1(=B178) (Georgopoulos, C., Hendrix, R. W., Casjens, S. R. & Kaiser, A. D. (1973) J. Mol. Biol. 76, 45-60), AI90 (ΔgroEL::kan^{R}) [pBAD-EL] (Ivic, A., Olden, D., Wallington, E. J. & Lund, P. A. (1997) Gene 194, 1-8), and TG1 (Gibson, T. J. (1984) Ph.D. thesis, University of Cambridge, U.K). Bacteriophage λb₂cI (Zeilstra-Ryalls, J., Fayet, O., Baird, L. & Georgopoulos, C. (1993) J. Bacteriol. 175, 1134-1143) was used according to standard methods (Arber, W., Enquist, L., Hohn, B., Murray, N. E. & Murray, K. (1983) in Lambda II., ed. R. W. Hendrix, J. W. r., F. W. Stahl and R. A. Weisberg (Cold Spring Harbor Laboratory, Cold Spring Harbor, N.Y.), pp. 433-466); plaque formation was assayed at 30° C. T4_{D0}, a derivative of bacteriophage T4 (Zeilstra-Ryalls, J., Fayet, O., Baird, L. & Georgopoulos, C. (1993) J. Bacteriol. 175, 1134-1143), was used according to standard methods (Karam, J. D. (1994) Molecular biology of bacteriophage T4. (American Society for Microbiology, Washington, D.C.)); plaque formation was assayed at 37° C.

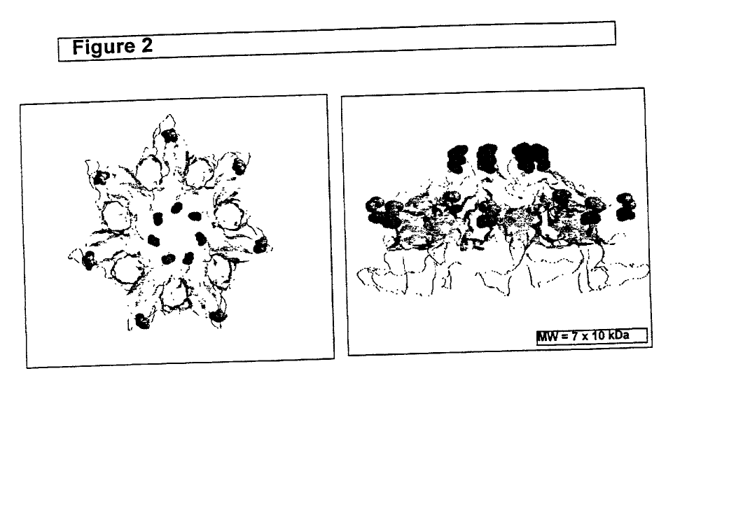

Plasmid constructions. Standard molecular biology procedures were used (Sambrook, J., Fritsch, E. F. & Maniatis, T. (1989) Molecular Cloning. A Laboratory Manual (Cold Spring Harbor Laboratory Press, N.Y.)). The schematic organisation of the plasmids used in this study is represented FIG. 2. Gp31 gene was PCR (polymerase chain reaction) amplified using two oligonucleotides 5′-C TTC AGA CAT ATG TCT GAA GTA CAA CAG CTA CC-3′ and 5′-TAA CGG CCG TTA CTT ATA AAG ACA CGG AAT AGC-3′ producing a 358 bp DNA using pSV25 (van der Vies, S., Gatenby, A. & Georgopoulos, C. (1994) Nature 368, 654-656) as template. The DNA sequence of a part of the mobile loop of Gp31 (residues 25 to 43) was removed by PCR, as described (Hemsley, A., Arnheim, N., Toney, M. D., Cortopassi, G. & Galas, D. J. (1989) Nucleic Acids Res. 17, 6545-6551), using oligonucleotides 5′-GGA GAA GTT CCT GAA CTG -3′ and 5′-GGA TCC GGC TTG TGC AGG TTC-3′, creating a unique BamH I site (bold characters). GroEL gene minichaperone (corresponding to the apical domain of GroEL. residues 191 to 376; (Zahn, R., Buckle, A. M., Perret, S., Johnson, C. M. J., Corrales, F. J., Golbik, R. & Fersht, A. R. (1996) Proc. Natl. Acad. Sci. U.S.A. 93, 15024-15029)) was amplified by PCR using oligonucleotides, containing a BamH I site (underlined), 5′-TTC GGA TCC GAA GGT ATG CAG TTC GAC C-3′ and 5′-GTT GGA TCC AAC GCC GCC TGC CAG TTT C-3′ and cloned into the unique BamH I site of pRSETA-Gp31Δloop vector, inserting minichaperone GroEL(191-376) in frame into Gp31Δloop sequence. The single ring GroEL_{SR1} mutant contains four amino acid substitutions (R452E, E461A, S463A, and V464A) into the equatorial interface of GroEL, which prevent the formation of double rings (Weissman, J. S., Hohl, C. M., Kovalenko, O., Kashi, Y., Chen, S., Braig, K., Saibil, H. R., Fenton, W. A. & Horwich, A. L. (1995) Cell 83, 577-587). The corresponding mutations were introduced into groEL by PCR (Hemsley, A., Arnheim, N., Toney, M. D., Cortopassi, G. & Galas, D. J. (1989) Nucleic Acids Res. 17, 6545-6551) using oligonucleotides 5′-TGA GTA CGA TCT GTT CCA GCG GAG CTT CC-3′ and 5′-ATT GCG GCG AAG CGC CGG CTG CTG TTG CTA ACA CCG-3′ and pRSETA-Eag I GroEL or GroESL vectors (Chatellier, J., Hill, F., Lund, P. & Fersht, A. R. (1998) Proc. Natl. Acad. Sci. U.S.A. 95, 9861-9866) as template; silent mutations, in respect to the codon usage in E. coli, create a unique Mfe I (bold characters) and Nae I (underlined). GroEL(E191G; groEL44 allele) gene was PCR amplified from E. coli SV2 strain (Zeilstra-Ryalls, J., Fayet, O., Baird, L. & Georgopoulos, C. (1993) J. Bacteriol. 175, 1134-1143) using two oligonucleotides 5′-T AGC TGC CAT ATG CCA GCT AAA GAC CTA AAA TTC GG-3′ and 5′-ATG TAA CGG CCG TTA CAT CAT GCC GCC CAT GCC ACC-3′ producing a 1,659 bp DNA with unique sites for Nde I and Eag I (underlined). The different genes were subcloned into the unique Nde I and Eag I unique sites of pACYC 184, pJC and pBAD30 (Guzman, L.-M., Belin, D., Carson, M. J. & Beckwith, J. (1995) J. Bacteriol. 177, 4121-4130) vectors (Chatellier, J., Hill, F., Lund, P. & Fersht, A. R. (1998) Proc. Natl. Acad. Sci. U.S.A. 95, 9861-9866). A colony-based PCR procedure was used to identify the positive clones (Chatellier, J., Mazza, A., Brousseau, R. & Vemet, T. (1995) Analyt. Biochem. 229, 282-290). PCR cycle sequencing using fluorescent dideoxy chain terminators (Applied Biosystems) were performed and analysed on an Applied Biosystems 373A Automated DNA. All PCR amplified DNA fragments were sequenced after cloning.

Proteins expression purification and characterisation. The GroE proteins, ˜57.5 kDa GroEL and ˜10 kDa GroES, were expressed and purified as previously described (Chatellier, J., Hill, F., Lund, P. & Fersht, A. R. (1998) Proc. Natl. Acad. Sci. U.S.A. 95, 9861-9866; Corrales, F. J. & Fersht, A. R. (1996) Folding & Design 1, 265-273). GroEL_{SR1} mutant was expressed and purified using the same procedure used for the wild-type GroEL; GroEL_{SR1} mutant was separated from emdogenous wild-type GroEL by ammonium sulphate precipitation at 30% saturation. GroEL(E191G) protein was expressed by inducing the P_{BAD} promoter of pBAD30 based vector with 0.2% arabinose in E. coli SV2 strain (Zeilstra-Ryalls, J., Fayet, O., Baird, L. & Georgopoulos, C. (1993) J. Bacteriol. 175, 1134-1143). Purification was performed essentially as described (Corrales, F. J. & Fersht, A. R. (1996) Folding & Design 1, 265-273). Residual peptides bound to GroEL proteins were removed by ion-exchange chromatography on a MonoQ column (Pharnacia Biotech.) in presence of 25% methanol. The over-expression of histidine-tagged (short histidine tail; sht)-minichaperone GroEL(191-376) in E. coli C41(DE3) cells and, the purification and the removal of sht by thrombin cleavage were carried out essentially as previously described (Zahn, R., Buckle, A. M., Perret, S., Johnson, C. M. J., Corrales, F. J., Golbik, R. & Fersht, A. R. (1996) Proc. Natl. Acad. Sci. U.S.A. 93, 15024-15029). Gp31 proteins wild-type (˜12 kDa), Δloop (˜10.4 kDa) and MC₇ (˜30.6 kDa), were expressed by inducing the T7 promoter of pRSETA-Eag I based vectors with isopropyl-β-D-thiogalactoside (IPTG) in E. coli C41(DE3) (Miroux, B. & Walker, J. E. (1996) J. Mol. Biol. 260, 289-298) overnight at 25° C. Purification procedures were essentially as described (van der Vies, S., Gatenby, A. & Georgopoulos, C. (1994) Nature 368, 654-656; Castillo, C. J. & Black, L. W. (1978) J. Biol. Chem. 253, 2132-2139). Ammonium sulphate precipitation (only 20% saturation for Δloop; 30 to 70% saturation for wild-type and MC₇) was followed by ion-exchange chromatography on a DEAE-Sepharose column (Pharmacia Biotech.). Gp31 proteins were eluted with a 0-0.5 M NaCl gradient in 20 mM Tris-HCl, 1 mM EDTA, 1 mM β-mercaptoethanol, pH 7.5; Δloop and MC₇ eluted between 0.32-0.44 and 0.38-0.48 mM NaCl, respectively.

Gp31 proteins were further purified by gel filtration chromatography on a Superdex™ 200 (Hiload 26/10) column (Pharmacia Biotech.) equilibrated with 100 mM Tris-HCl, pH 7.5 and, dialysed against and stored in 50 mM Tris-HCl, 0.1 mM EDTA, 1 mM β-mercaptoethanol, pH 7.5. Proteins were analysed by electrospray mass spectrometry. Protein concentration was determined by absorbance at 276 nm using the method of Gill & von Hippel (Gill, S. C. & von Hippel, P. H. (1989) Analyt. Biochem. 182, 319-326) and confirmed by quantitative amino acid analysis.

Constitutive expression under the control of the tetracycline-resistance gene promoter/operator was obtained using the high copy-number pJC vectors (Chatellier, J., Hill, F., Lund, P. & Fersht, A. R. (1998) Proc. Natl. Acad. Sci. U.S.A. 95, 9861-9866). pBAD30 vector allows inducible expression with 0.2-0.5% arabinose controlled by the P_{BAD} promoter and its regulatory gene, araC (Guzman, L.-M., Belin, D., Carson, M. J. & Beckwith, J. (1995) J. Bacteriol. 177, 4121-4130). The level of expression of MC₇ was analysed by 15% sodium dodecyl sulphate polyacrylamide gel electrophoresis (SDS-PAGE) under non-reducing conditions followed by Western blotting as described (Chatellier, J., Hill, F., Lund, P. & Fersht, A. R. (1998) Proc. Natl. Acad. Sci. U.S.A. 95, 9861-9866).

Molecular weight determination by analytical gel filtration chromatography and analytical ultracentrifugation. One hundred μl aliquots of protein (1 mg.mL⁻¹) were loaded onto a Superdex™ 200 (HR 10/30) column (Pharmacia Biotech.) equilibrated with 50 mM Tris-HC1, 150 mM NaCl, pH 7.5 at 0.5 mL.min⁻¹ at 20° C. The column was calibrated using gel filtration standards from Pharmacia Biotech. (thyroglobulin, MW=669 kDa; ferritin, MW=440 kDa; aldolase, MW=158 kDa; ovalbulmin, MW=45 kDa; chymotrypsinogen MW=25 kDa; RNase, MW=13 kDa). Molecular weights were determined by logarithmic interpolation.

Sedimentation analysis was performed in 50 mM Tris-HCl, 2.5 mM DTE (dithio-erythritol), pH 7.2 at 20° C. with protein concentration in the range 45-300 μM, scanning at 280 nm, with a Beckman XL-A analytical ultracentrifuge, using an An-60Ti rotor. Sedimentation equilibrium experiments were at 10,000 rev.min⁻¹ with overspeeding at 15,000 rev.min⁻¹ for 6 hours to speed the attainment of equilibrium. Scans were taken at intervals of 24 hours, until successive scans superimposed exactly, when the later scan was taken as being operationally at equilibrium. To evaluate the apparent average molecular weight, data were fitted by non-linear regression.

Circular dichroism spectroscopy (CD). Far UV (200-250 nm)-CD spectra at 25° C. were measured on a Jasco J720 spectropolarimeter interfaced with a Neslab PTC-348WI water bath, using a thermostatted cuvette of 0.1 cm path length. Spectra are averages of 10 scans and were recorded with a sampling interval of 0.1 nm. Thermal denaturation was carried out from 5-95° C. at a linear rate of 1° C.min⁻¹ and monitored at 222 nm. The reversibility was checked after incubation at 95° C. for 20 min and cooling to and equilibration at 5° C. The protein concentration was 45 μM in 10 mM sodium phosphate buffer pH 7.8, 2.5 mM DTE (dithioerythrol).

GroES binding and competition assays by ELISA (enzyme-linked immunosorbant assay). Proteins were coated onto plastic microtitre plates (Maxisorb, Nunc) overnight at 4° C. at a concentration of 10 μg/mL in carbonate buffer (50 mM NaHCO₃, pH 9.6). Plates were blocked for 1 hour at 25° C. with 2% Marvel in PBS (phosphate buffered saline: 25 mM NaH₂PO₄, 125 mM NaCl, pH 7.0). GroES, at 10 μg/mL in 100 μL of 10 mM Tris-HCl, 200 mM KCl, pH 7.4, were bound at 25° C. for 1 hour. Bound GroES were detected with rabbit anti-GroES antibodies (Sigma) followed by anti-rabbit immunoglobulins horseradish peroxidase conjugated antibodies (Sigma).

A peptide corresponding to the mobile loop of GroES (residues 16 to 32, numbered as in Hemmingsen, S. M., Woolford, C., van, d. V. S., Tilly, K., Dennis, D. T., Georgopoulos, C. P., Hendrix, R. W. & Ellis, R. J. (1988) Nature 333, 330-334) was synthesised as described (Chatellier, J., Buckle, A. M. & Fersht, A. R. (1999) J. Mol. Biol., in press). The inhibition of the binding of MC₇ proteins by the free peptide was analysed by ELISA, essentially as above, by adding different concentrations (between 10,000 to 0.1 μM) of free peptide solved in 0.1% TFA solution to 1 μg of proteins prior incubation to coated GroES proteins (10 μg/mL). GroEL molecules were detected with rabbit anti-GroEL antibodies (Sigma) followed by anti-rabbit immunoglobulins horseradish peroxidase conjugate antibodies (Sigma). ELISAs were developed with 3′,3′,5′,5′-tetramethylbenzidine (TMB, Boehringer Mannheim). Reactions were stopped with 50 μl of 1M H₂SO₄ after 10 min and readings taken by subtracting the O.D._{650 nm} from the O.D._{450 nm}.

Anti-GroEL antibodies binding by ELISA. The same amount of proteins (1 μg) were coated as described above. GroEL molecules were detected with either (i) rabbit anti-GroEL horseradish peroxidase conjugate antibodies (9 mg/mL; Sigma) or (ii) rabbit anti-GroEL antibodies (11.5 mg/mL; Sigma) followed by anti-rabbit immunoglobulins horseradish peroxidase conjugate antibodies (Sigma). ELISAs were developed as described above.

In vitro refolding experiments. Refolding assays of pig heart mitochondrial malate dehydrogenase (mtMDH; Boehringer-Mannheim) and aggregation protection were carried out essentially as described (Peres Ben-Zvi, A. P., Chatellier, J., Fersht, A. R. & Goloubinoff, P. (1998) Proc. Natl. Acad. Sci. U.S.A. 95, 15275-15280). The concentrations of MC₇ used were between 8-16 μM (reporter to protomer).

In vivo complementation experiments. Complementation experiments were performed by transforming electro-competent SV2 or SV6 cells with the pJC series of expression vectors and plating an aliquot of the transformation reactions directly at 43° C. The percentage of viable cells relative to the growth at 30° C. was determined. A representative number of clones which grew at 43° C. were incubated in absence of any selective markers at permissive temperature. After prolonged growth the loss of the pJC plasmids and the ts phenotype were verified. Each experiment was performed in duplicate. Plasmids carrying no groE genes or encoding the GroE proteins were used as negative or positive controls, respectively.

P1 transduction (Miller, J. H. (1972) Experiments in Molecular Genetics (Cold Spring Harbor, N.Y.)), using strain AI90 (ΔgroEL::kan^{R}) [pBAD-EL] as donor (Ivic, A., Olden, D., Wallington, E. J. & Lund, P. A. (1997) Gene 194, 1-8), was used to delete the groEL gene of TG1 cells transfected by the different pJC vectors. Transductants were selected on LB plates containing 10 μg/mL of kanamycin at 37° C. Approximately 25 colonies were transferred onto plates containing kanamycin at 50 μg/mL. After incubation for 24 h at 37° C., colonies that grew were screened by PCR as described.

AI90 (ΔgroEL::kan^{R}) [pBAD-EL] cells were transformed with the pJC vector series. Transformants were selected at 37° C. on LB supplemented with 50 μg.ml⁻¹ of kanamycin, 120 μg.mL⁻¹ of ampicillin, 25 μg.mL¹ of chloramphenicol and 0.2% L(+)arabinose. Depletion of GroEL protein was analysed at 37° C. by plating the same quantity of AI90 [pBAD-EL+pJC vectors] cells on LB plates containing 1% D(+)glucose or various amount of arabinose.

Each experiment was performed in triplicate. Plasmids carrying no groE genes or encoding the GroE proteins were used as negative or positive controls, respectively.

Effect on Lorist6 replication of over-expressing of MC₇. The effect of over-expressing Gp31 proteins from pJC vector series on the replication of the bacteriophage λ origin vector, Lorist6 (Gibson, T. J., Rosenthal, A. & Waterston, R. H. (1987) Gene 53, 283-286) in TG1 (Gibson, T. J. (1984) Ph.D. thesis, University of Cambridge, U.K) or SV1 (Georgopoulos, C., Hendrix, R. W., Casjens, S. R. & Kaiser, A. D. (1973) J. Mol. Biol. 76, 45-60) cells was determined essentially as described (Chatellier, J., Hill, F., Lund, P. & Fersht, A. R. (1998) Proc. Natl. Acad. Sci. U.S.A. 95, 9861-9866).

### 2. Example 1

Gp31 Protein as a Scaffold for Displaying Heptameric GroEL Minichaperone.

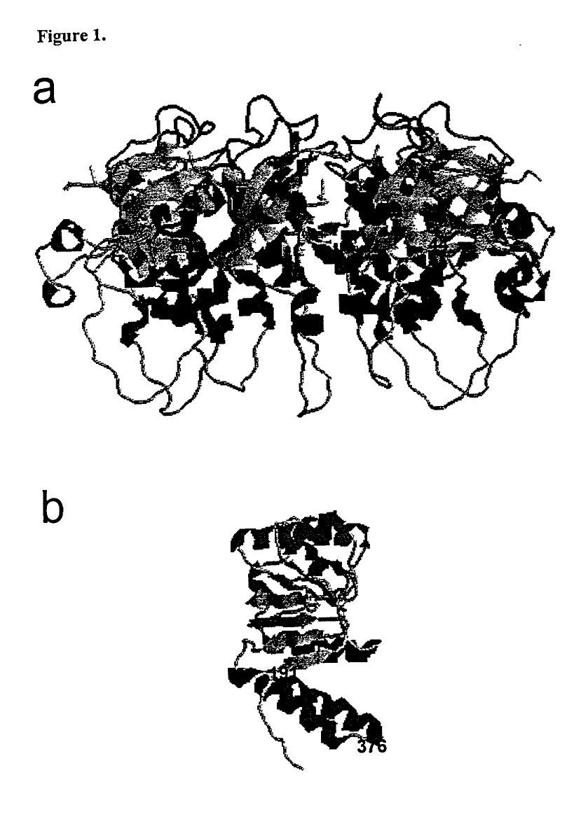

We describe a scaffold on which any polypeptide may be hung; as a result, the polypeptide is oligomerised. The scaffold is the bacteriophage T4 Gp31 (gene product) heptamer. The monomeric protein is 12 kDa, but it spontaneously forms a stable heptameric structure (90 kDa) of which the three-dimensional structure is known from X-ray crystallography (Hunt, J. F., van der Vies, S., Henry, L. & Deisenhofer, J. (1997) Cell 90, 361-371). This illustrates that a highly mobile polypeptide loop (residues 25 to 43; Chatellier, J., Mazza, A., Brousseau, R. & Vemet, T. (1995) Analyt. Biochem. 229, 282-290) projects from each subunit (FIG. 1). The basis of the method is the substitution of this loop by a chosen peptide sequence.

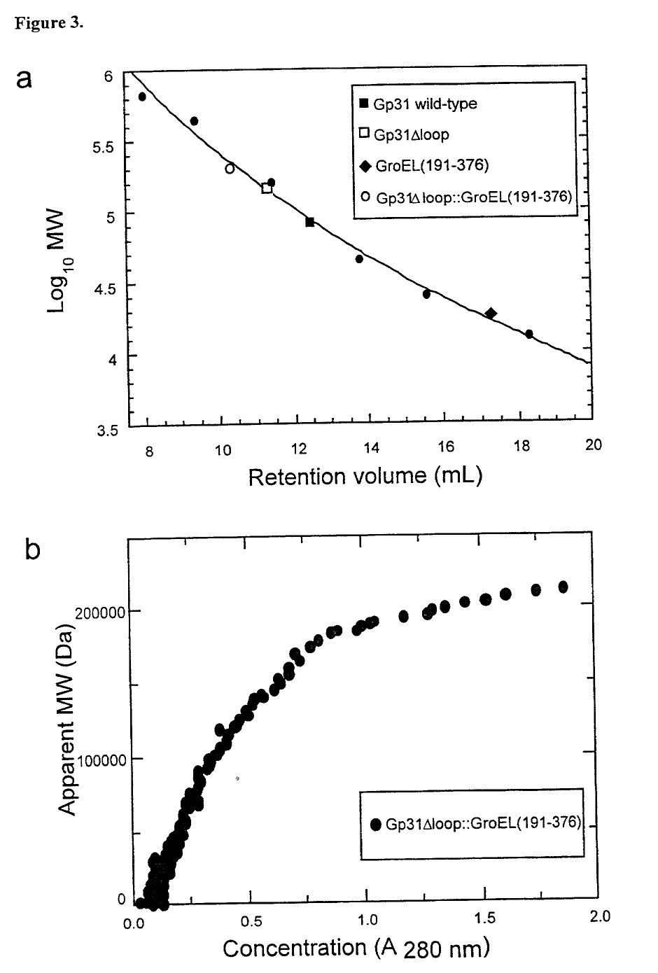

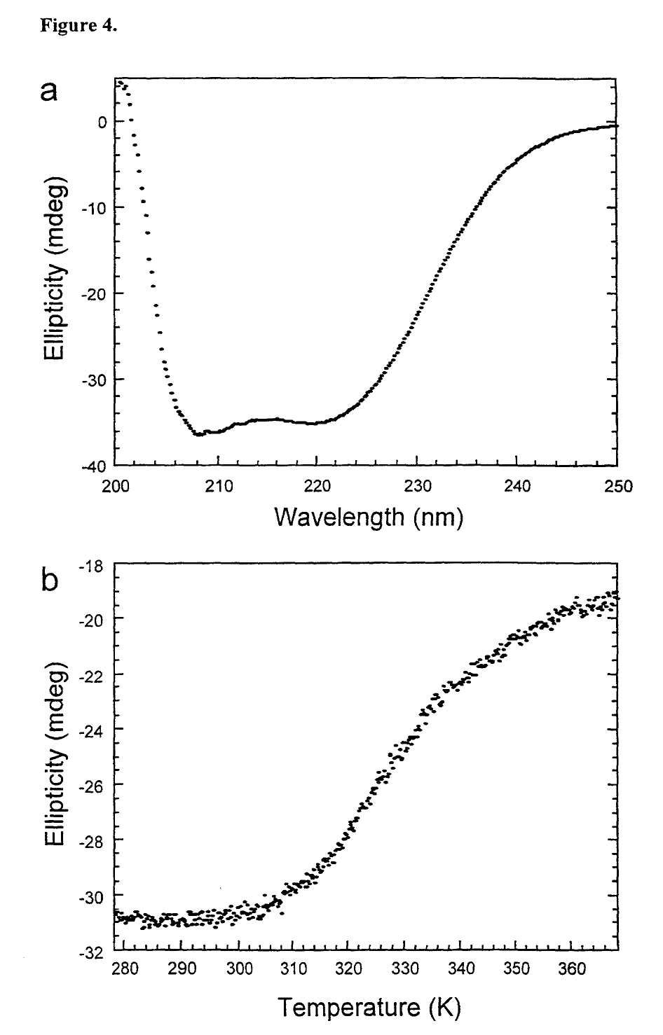

In an effort to increase the avidity of minichaperones for substrates, and consequently to improve their chaperonin-facilitated protein folding, we generated the fusion protein, Gp31Δloop::GroEL(191-376) (hereafter named MC₇), where the mobile loop of Gp31 was replaced by the sequence of minichaperone GroEL (residues 191 to 376) (FIG. 2). MC₇ was cloned downstream of the T7 promoter of pRSETAsht-Eag I vector (Chatellier, J., Hill, F., Lund, P. & Fersht, A. R. (1998) Proc. Natl. Acad. Sci. U.S.A. 95, 9861-9866). After sonication, the soluble and insoluble fractions of IPTG-induced transfected C41(DE3) cells (Miroux, B. & Walker, J. E. (1996) J. Mol. Biol. 260, 289-298) were analysed by SDS-PAGE. Most of MC₇ was present in the insoluble fraction. Insoluble material dissolved in 8 M urea was efficiently refolded by dialysis at 4° C. MC₇ was purified by ion-exchange and gel filtration chromatography. MC₇ was over-expressed in C41(DE3) cells to give 0.25-0.5 g purified protein per L of culture. Purified MC₇ coincided to seven 30.6 kDa subunits of Gp31Δloop::GroEL(191-376) as determined by analytical size exclusion chromatography (FIG. 3a) and analytical ultracentrifugation (FIG. 3b); Gp31Δloop corresponds to a tetra-decamer (14 subunits). The introduction of a foreign polypeptide in the Gp31 scaffold does do prevent its oligomerisation ability. The electronic microscopy studies of MC₇ revealed views that correspond to front views of oligomers with a diameter close to the one of GroEL (J. L. Carrascosa, J. C. & A. R. F., unpublished). The circular dichroism spectrum of MC₇ indicated significant α-helical structure (FIG. 4a). The thermal unfolding monitored by far UV-CD was reversible although more than one transition exist (FIG. 4b).

Bacterial GroES or the human mitchodrial Hsp10 homologous oligomerisable scaffolds have been also successfully used to oligomerise polypeptides displayed in their mobile loops.

### 3. Example 2

Binding to Heptameric Bacterial Co-chaperonin, GroES.

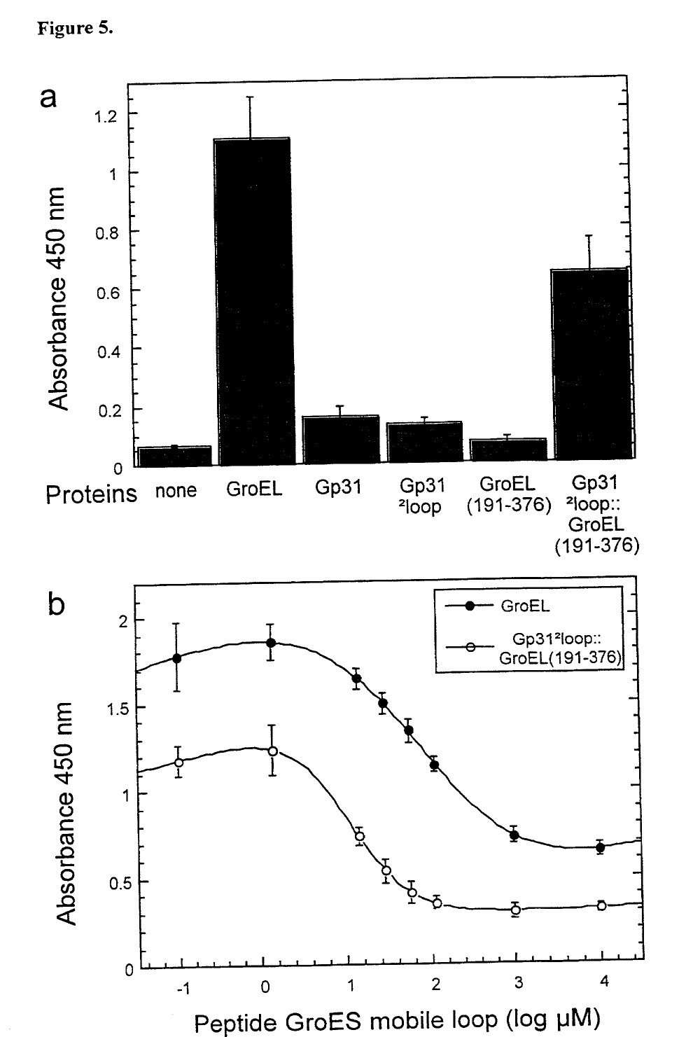

The functionality of MC₇ was examined for binding to GroES, since the interaction between GroEL and GroES is known to be less favourable for one monomer than for the heptamer. MC₇ bound specifically to GroES, conversely monomeric minichaperone GroEL(191-376) did not detectably bind the bacterial co-chaperonin (FIG. 5a).

The ability of a synthetic peptide corresponding to residues 16 to 32 of GroES mobile loop to displace bound GroES from MC₇ was tested by competition ELISA. The synthetic GroES mobile loop peptide did inhibit the binding of MC₇ with an IC₅₀ of 10 μM compared to 100 μM for GroEL (FIG. 5b). The apparent dissociation constant for the formation of the GroEL-GroES complex is low (10⁻⁶ M), which is compatible with cycling of GroES on and off GroEL during chaperonin-assisted folding. On the other hand, GroEL_{SR1} (Weissman, J. S., Rye, H. S., Fenton, W. A., Beechem, J. M. & Horwich, A. L. (1996) Cell 84, 481-490) is unable to release GroES in the absence of signal transmitted via the binding of ATP to an adjacent ring. The 10-fold decrease of the affinity of MC₇ for GroES may be sufficient for multiple binding and release cycles.

### 4. Example 3

Binding to Antibodies.

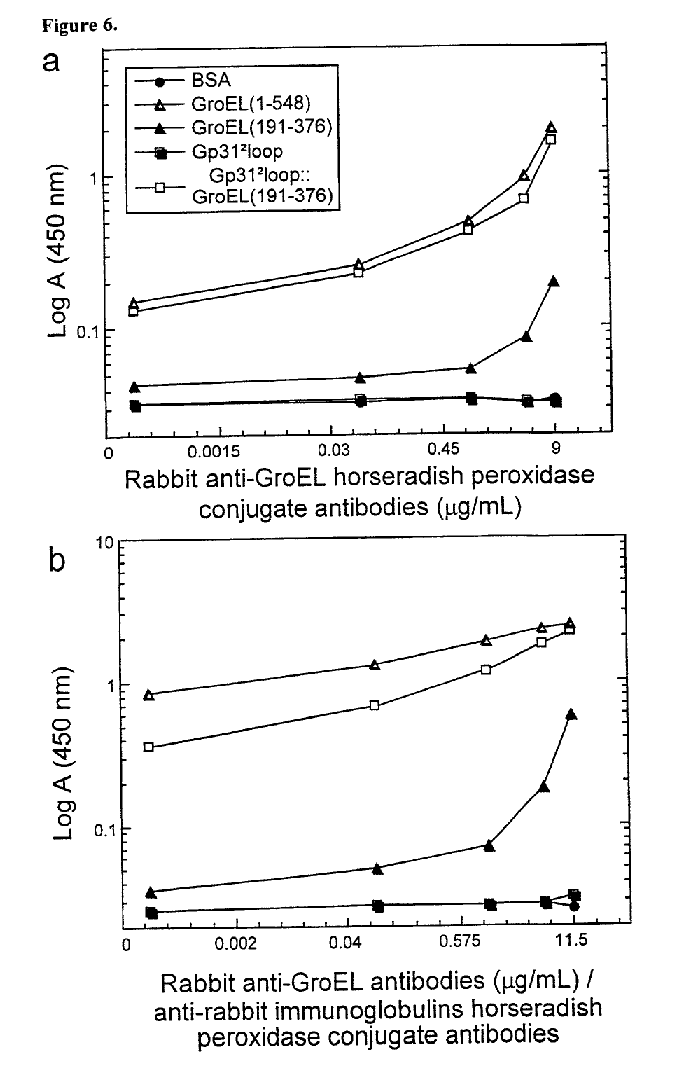

A major application of oligomerisable scaffolds is the conversion of the hung polypeptides to antigens. The oligomerisation improves both detection of antibodies against, and the induction of antibodies to, such antigens. Indeed, the avidity effect of the heptameric structure of MC₇ was confirmed by analysing the binding of antibodies specific to GroEL (FIG. 6); comparable detection levels were observed for GroEL and MC₇ at the different concentrations of antibodies used. In addition, using affinity panning of immobilised MC₇ for a large library of bacteriophages (“phage display”) that display single-chain Fv (scFv) antibodies fragments, we selected recombinant monoclonal scFvs that recognised only and specifically GroEL(191-376), and not the scaffold, Gp31Δloop (P. Wang, J. C., G. Winter & A. R. F., unpublished). This demonstrates the advantages of displaying polypeptides in a scaffold for immunisation purposes.

### 5. Example 4

In vitro Activity of MC₇.

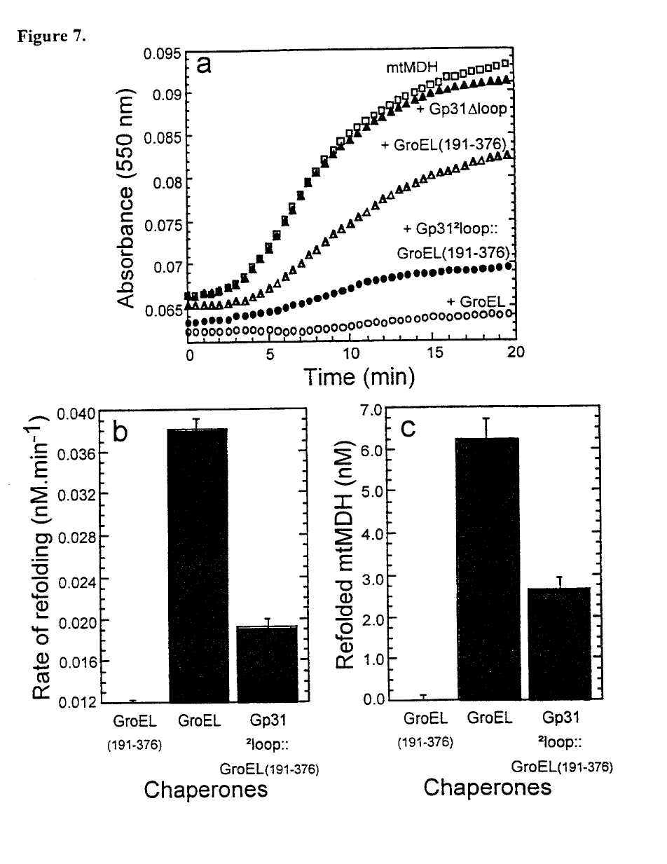

In vitro, heat- and dithiothreitol-denatured mitochondrial malate dehydrogenase (mtMDH) refolds in high yield only in the presence of GroEL, ATP, and the co-chaperonin GroES (Peres Ben-Zvi, A. P., Chatellier, J., Fersht, A. R. & Goloubinoff, P. (1998) Proc. Natl. Acad. Sci. U.S.A. 95, 15275-15280). Monomeric minichaperone GroEL(191-376) binds denatured mtMDH, protecting is aggregation (FIG. 7a) but, it is ineffective in enhancing the refolding rate (FIG. 7b). Conversely, MC₇, which protect further denatured mtMDH from aggregation (FIG. 7a) is active in refolding denatured mtMDH (FIG. 7a) with a rate of 0.02 nM.min⁻¹, compared to 0.04 nM.min⁻¹ for wild-type GroEL alone (FIG. 7b). After 120 min, the yield of refolded mtMDH by MC₇ is about 2.5-3 nM, compared to 6 nM of enzyme rescued by wild-type GroEL (FIG. 7c). Although saturating concentration of GroES (4 μM) does increase about 3- to 5-fold the rates at the beginning of the refolding reaction, a 10-fold decrease of the final yield was observed; indicating the absence of multiple cycles of binding and release of GroES to MC₇ (data not shown). Nevertheless, MC₇ is more efficient than GroEL_{SR1} mutant (Llorca, O., Pérez-Pérez, J., Carrascosa, J., Galan, A., Muga, A. & Valpuesta, J. (1997) J. Biol. Chem. 272, 32925-32932; Nielsen, K. L. & Cowan, N. J. (1998) Molecular Cell 2, 1-7; this study); remarkably, MC₇ is only 2-fold less active than wild-type GroEL in refolding a non-pernissive substrate in vitro. This demonstrates the advantages of oligomerised peptides in increasing avidity of binding.

### 6. Example 5

In vivo Complementation of Thermosensitive groEL Mutant Alleles at 43° C.

We sought complementation of two thermosensitive (ts) groEL mutants of E. coli at 43° C. E. coli SV2 has the mutation Glu191→Gly in GroEL corresponding to groEL44 allele, while SV6 carries the EL673 allele, which has two mutations, Gly173→Asp and Gly337→Asp. Complementation experiments were performed by transforming the thermosensitive (ts) E. coli strains SV2 or SV6 with the pJC series of expression vectors vector (Chatellier, J., Hill, F., Lund, P. & Fersht, A. R. (1998) Proc. Natl. Acad. Sci. U.S.A. 95, 9861-9866) and plating an aliquot of the transformation reaction directly at 43° C. Subsequently, plasmids pJC from a representative number of individual clones growing at 43° C. were lost in the absence of continued chloramphenicol selection. Nearly all (≧95%) the cured clones were thermosensitive at 43° C. indicating the absence of recombination events for the reconstitution of wild-type groEL gene. The results obtained are qualitatively similar to those previously described (Chatellier, J., Hill, F., Lund, P. & Fersht, A. R. (1998) Proc. Natl. Acad. Sci. U.S.A. 95, 9861-9866). Only minichaperone sht-GroEL(193-335) complements the defect in SV2. The defective groEL in SV6 was complemented by expression of minichaperone sht-GroEL(191-345), and less well by sht-GroEL(193-335). Conversely, MC₇ and GroEL_{SR1} complement both temperature-sensitive E. coli groEL44 and groEL673 alleles at 43° C. (Table 1). Colony-forming units were not observed for either strain at 43° C. with vectors either lacking inserts (pJCsht) or lacking GroEL(191-376) (pJCGp31Δloop).

It has been suggested the higher stability of shortest minichaperone sht-GroEL(193-335) could be responsible for the complementation of groEL44 mutant allele. To test this eventuality, we purified GroEL(E191G; groEL44 allele) mutant and compared its thermal stability with the wild-type GroEL. We found no difference in stability between the mutant and the wild-type proteins in presence or absence of ATP. In addition, highly stable functional mutants of GroEL (193-345) do not complement, as the parental minichaperone (Table 1), the defects in SV2 or even SV6. We concluded the thermal stability of minichaperone is not accountable for the complementation of groEL defects.

TABLE 1
Relative colony forming ability of transformed ts
groEL44 or groEL673 E. coli strains at 43° C.

GroEL strains

| Plasmids pJC | SV2 groEL44 | SV6 groEL673 |
|---|---|---|
| short his tag (sht) (ES⁻, EL⁻) | <10⁻⁴ | <10⁻⁴ |
| GroES (1-97) | 5 × 10⁻³ | <10⁻⁴ |
| Gp31 (1-111) | 0.5 × 10⁻³ | <10⁻⁴ |
| Gp31 Δloop | <10⁻⁴ | <10⁻⁴ |
| GroEL (1-548) | 1 | 1 |
| GroES-EL | 1 | 1 |
| sht-GroEL (191-376) | <10⁻⁴ | <10⁻⁴ |
| sht-GroEL (191-345) | 0.01-0.02 | 0.07-0.09 |
| sht-GroEL (193-335) | 0.05-0.09 | 0.03-0.05 |
| Gp31Δ::GroEL (191-376) | 0.15-0.2 | 0.1 |

### 7. Example 6

In vivo Complementation at 37° C.

The effects of MC₇ on the growth at 37° C. of a strain of E. coli in which the chromosomal groEL gene had been deleted were analysed in two ways. First, we attempted to delete the groEL gene of TG1 which had been transformed with the different pJC MC₇ vector by P1 transduction. However, no transductants could be obtained where the groEL gene had been deleted, unless intact GroEL was expressed from the complementing plasmid. This is consistent with the known essential role of GroEL. Second, we analysed the complementation of AI90 (ΔgroEL::kan^{R}) [pBAD-EL] E. coli strain. In this strain, the chromosomal groEL gene has been deleted and GroEL is expressed exclusively from a plasmid-bome copy of the gene which can be tightly regulated by the arabinose P_{BAD} promoter and its regulatory gene, araC. AraC protein acts as either a repressor or an activator depending on the carbon source used. PBAD is activated by arabinose but repressed by glucose (Guzman, L.-M., Belin, D., Carson, M. J. & Beckwith, J. (1995) J. Bacteriol. 177, 41214130). The AI90 [pBAD-EL] cells can not grow on medium supplemented with glucose at 37° C. (Ivic, A., Olden, D., Wallington, E. J. & Lund, P. A. (1997) Gene 194, 1-8). As minichaperones (Chatellier, J., Hill, F., Lund, P. & Fersht, A. R. (1998) Proc. Natl. Acad. Sci. U.S.A. 95, 9861-9866.), MC₇ was unable to suppress this groEL growth defect (Table 2). We then determined whether MC₇ could supplement low levels of GroEL from transfected AI90 [pBAD-EL]. At 0.01% arabinose, cells transfected with pJC expressing sht alone, Gp31Δloop or sht-GroEL(191-376), showed little colony forming ability (lees than 5%). But those containing pJC MC₇ produced about 30% of the number produced in the presence of 0.2% arabinose. Thus, pJC MC₇, but not pJCGroEL_{SR1}, significantly supplements depleted levels of GroEL., about twice as pJC sht-GroEL(193-335) (Chatellier, J., Hill, F., Lund, P. & Fersht, A. R. (1998) Proc. Natl. Acad. Sci. U.S.A. 95, 9861-9866).

TABLE 2
Plating ability of transformed AI90 (ΔgroEL::kan^{R}) [pBADEL]
E. coli strain at 37° C. in presence of different amount of arabinose.

% L(+)arabinose

| Plasmids pJC | 0.15 | 0.10 | 0.01 | 0.00 |
|---|---|---|---|---|
| short his tag (sht) (ES⁻, EL⁻) | ++ | + | +/− | − |
| Gp31Δloop | ++ | + | +/− | − |
| GroEL (1-548) | +++ | +++ | +++ | +++ |
| sht-GroEL (191-376) | ++ | + | +/− | − |
| Gp31Δ::GroEL (191-376) | +++ | +++ | + | − |

+++, growth identical to that in presence of 0.2% L(+)arabinose (100%), in terms of both number and size;
++, about 50% of the colonies relative to that in presence of 0.2% L(+)arabinose;
+, about 30% of the colonies;
+/− ≦5% of the colonies and size reduced relative to that in presence of 0.2% L(+)arabinose;
−, no visible colonies.

### 8. Example 7

Effect on Bacteriophages λ and T4 Growth of Over-expressing MC₇.

Bacteriophage λ requires the chaperonins GroES and GroEL for protein folding during morphogenesis; bacteriophage T4 requires GroEL and Gp31, the latter being encoded by the bacteriophage genome (Zeilstra-Ryalls, J., Fayet, O. & Georgopoulos, C. (1991) Annu. Rev. Microbiol. 45, 301-325). Nine groE alleles which fail to support λ growth have been sequenced (Zeilstra-Ryalls, J., Fayet, O., Baird, L. & Georgopoulos, C. (1993) J. Bacteriol. 175, 1134-1143). We examined the ability of MC₇, over-expressed from the constitutive tet promoter on a high-copy number vector (see FIG. 2), to complement three mutant groEL alleles for plaque formation by λ (b₂cI) at 30° C. (Table 3) and T4 at 37° C. (Table 4).

The groE operon was named for its effects on the E protein of λ (Georgopoulos, C., Hendrix, R. W., Casjens, S. R. & Kaiser, A. D. (1973) J. Mol. Biol. 76, 45-60). Although heat induction of the groE operon has been shown to decrease burst size of λ bacteriophage in E. coli (Wegrzyn, A., Wegrzyn, G. & Taylor, K. (1996) Virology 217, 594-597). In contrast, we showed that the over-expression of GroEL alone, which resulted in slower growth of the bacteria, suffices to inhibit λ growth (Table 3). This effect was specific; over-expression of GroEL together with GroES caused only a four-fold drop in plaques. Over-expression of GroES alone had no effect. Minichaperone GroEL(191-376) had no effect on plaque counts in SVI (groE⁺). Conversely, over-expression of MC₇ prevents plaque formation by bacteriophage λ in SV1, but less markedly than GroEL (Table 3). It seems that the main effect of GroEL over-expression is mediated through the λ origin, which requires two proteins, O and P. As with GroEL, MC₇ (or GroEL_{SR1}) inhibit the replication of the Lorist6 plasmid which use the bacteriophage λ origin. The effect on Lorist6 shows that the unfoldase activity is also an essential part of GroEL activity in vivo. MC₇ and minichaperones possess both, un- and folding, activities. GroEL over-expression gives weak complementation of λ growth in SV2 (groEL44) and SV3 (groEL59; Ser201→Phe). MC₇ does not, but GroEL_{SR1} does complement any of the E. coli groEL mutant strains for bacteriophage λ growth at 30° C. (Table 3).

Bacteriophage T4 (T4_{D0}) also requires a functional groEL gene, but encodes a protein Gp31 which can substitute for GroES. The requirement for GroEL can be distinguished genetically from λ's requirement. Thus only two of the four groEL alleles fail to support T4 replication; these are also the two thermosensitive mutations EL44 and EL673 (Zeilstra-Ryalls, J., Fayet, O., Baird, L. & Georgopoulos, C. (1993) J. Bacteriol. 175, 1134-1143; Zeilstra-Ryalls, J., Fayet, O. & Georgopoulos, C. (1991) Annu. Rev. Microbiol. 45, 301-325). While over-expression of Gp31 allows T4 growth in all strains (only SV2 and SV6 strains normally do not allow T4 growth), over-expression of Gp31Δloop inhibits T4 replication. On the other hand, MC₇ does, as does GroEL_{SR1}, complement E. coli groEL mutant strains for bacteriophage T4 growth at 30° C. (Table 3).

### 9. Example 8

Single Ring Mutants GroEL_{SR1} or GroEL_{SR1} as Scaffolds

Surprisingly, over-expression of GroES demonstrates allele-specific complementation for λ and T4 of GroEL44 (Glu191→Gly) mutant (Tables 3 & 4). The effect is nevertheless incomplete; plaques on SV2 [pJCGroES] are invariably smaller than on SV1, or SV1 [pJCGroES]. The E191G single mutation blocks the assembly of the head structure of bacteriophage λ. A possible molecular basis for this allele-specificity lies in the nature of the groEL44 mutation. The substitution of Glu191→Gly in the hinge region between the intermediate and apical domains of GroEL presumably increases the flexibility of the hinge, and thereby, modulates a hinged conformational change in GroEL required for proper interaction with GroES. Indeed, the pivoting of the hinge region ensures proper interaction with GroES. For example, the mutant GroEL59 (Ser201→Phe in the same hinge region) in SV3 has low affinity for GroES. Over-expression of GroES will favour the formation of GroES-EL44 complex; we indeed also observed complementation of SV2 for thermosensitivity and bacteriophages growth by over-expressing GroEL44 mutant. Taking advantage of the GroES effect, we observed that GroEL minichaperones and MC₇ all reduce both plaque size and number but, like GroEL, do not completely eliminate them in SV2 [pBADGroES].

GroEL44, purified to homogeneity, is effective in refolding heat- and DTT-denatured mitochondrial malate dehydrogenase in presence of ATP and saturating concentration of GroES. Surprisingly, GroEL44 is as thermo-stable as the wild-type GroEL, indicating the mutation does not destabilise the overall conformation of the mutant. As anticipated from our in vivo genetic analysis, the affinity between GroEL44 and GroES is decreased at 37° C. and even more at higher temperature.

Our results suggest that the groEL44 mutation changes the distribution of GroEL subunits between apical domain-open and closed conformations. To allow GroEL_{SR1} to release GroES in the absence of signal transmitted via the binding of ATP to an adjacent ring, we introduced the Glu191→Gly mutation in GroEL_{SR1}, generating the GroEL_{SR2} mutant. GroEL_{SR2} is more efficient than MC₇ and even more than GroEL_{SR1} in vitro and in vivo.

TABLE 3
Growth of bacteriophage λ at 30° C. in
transformed wild-type and groEL mutant strains.

| | groEL strains | | | |
|---|---|---|---|---|
| | SV1 | SV2 | SV3 | SV6 |
| Plasmids pJC | (groEL⁺) | groEL44 | GroEL59 | groEL673 |
| short his tag (sht) | +++ | − | − | − |
| (ES⁻, EL⁻) | | | | |
| GroES (1-97) | +++ | +++ | − | − |
| Gp31 (1-111) | +++ | − | − | − |
| Gp31Δloop | +++ | − | − | − |
| GroEL (1-548) | − | + | ++ | +/− |
| sht-GroEL (191-376) | +++ | − | − | − |
| Gp31Δ::GroEL | + | − | − | − |
| (191-376) | | | | |

TABLE 4
Growth of bacteriophage T4 at 37° C. in
transformed wild-type and groEL mutant strains.

| | groEL strains | | | |
|---|---|---|---|---|
| | SV1 | SV2 | SV3 | SV6 |
| Plasmids pJC | (groEL⁺) | groEL44 | groEL59 | groEL673 |
| short his tag (sht) | +++ | − | +++ | − |
| (ES⁻, EL⁻) | | | | |
| GroES (1-97) | +++ | +++ | +++ | − |
| Gp31 (1-111) | +++ | +++ | +++ | +++ |
| Gp31Δloop | +/− | − | − | − |
| GroEL (1-548) | +++ | +++ | +++ | +++ |
| sht-GroEL (191-376) | +++ | − | +++ | − |
| Gp31Δ::GroEL | ++ | + | + | +/− |
| (191-376) | | | | |

+++, normal plaque-forming ability relative to wild-type groEL⁺ strain, in terms of both number and size;
++, 5-fold fewer plaques relative to wild-type groEL⁺ strain, or both;
+, 10-fold fewer plaques, or plaque size reduced relative to wild-type groEL⁺ strain, or both;
+/−, 10²-fold fewer plaques and plaque size reduced relative to wild-type groEL⁺ strain;
−, no visible plaques (<10⁻⁴).

### 10. Example 9

MC₇₂

A second oligomeric minichaperone polypeptide was constructed based on the GroES scaffold. This polypeptide, named MC₇₂, is GroESΔloop::GroEL(191-376).

Plasmid Constructions

Standard molecular biology procedures were used (Sambrook et al., 1989). The plasmid pRSETA encoding GroES gene has been described (Chatellier et al. 1998 In vivo activities of GroEL minichaperones. Proc. Nati. Acad. Sci. USA 95, 9861-9866). The GroES mutant Gly24Trp was generated by polymerase chain reaction (PCR), as described (Hemsley et al., 1989 A simple method for site-directed mutagenesis using the polymerase chain reaction. Nucl. Acids Res. 17, 6545-6551) using the template pRSETA encoding GroES (Chatellier et al., 1998) and the oligonucleotides 5′-C GGC TGG ATC GTT CTG ACC G-3′ and 5′-GC AGA TTT AGT TTC AAC TTC TTT ACG-3′, creating a Nae I site (bold characters).

The DNA sequence encoding a part of the mobile loop of GroES (residues 16 to 33) was removed by PCR, as described (Hemsley et al., 1989), using the oligonucleotides 5′-TCC GGC TCT GCA GCG G-3′ and 5′-TCC AGA GCC AGT TTC AAC TTC TTT ACG C-3′, creating a unique BamH I site (bold characters) and the vector pRSET A-GroESΔloop.

The GroEL minichaperone gene (corresponding to the apical domain of GroEL, residues 191 to 376; Zahn et al., 1996 Chaperone activity and structure of monomeric polypeptide binding domains of GroEL Proc. Nat. Acad. Sci. USA 93, 15024-15029) was amplified by PCR and cloned into the unique BamH I site of pRSETA-GroESΔloop vector, thus inserting the minichaperone GroEL(191-376) in-frame into the GroESΔloop sequence. These genes were subcloned into the unique Nde I and Eag I sites of pACYC184, pJC and pBAD30 vectors (Guzman et al., 1995, Tight regulation, modulation, and high level expression by vectors containing the pBAD promoter. J. Bacteriol. 177, 4121-4130; Chatellier et al., 1998). PCR cycle sequencing using fluorescent dideoxy chain terminators (Applied Biosystems) was performed and analysed on an Applied Biosystems 373A machine. All PCR amplified DNA fragments were sequenced after cloning.

Proteins expression, purification and characterisation. The GroES proteins, wild-type (˜10.4 kDa) and mutant Gly24Trp (˜10.5 kDa), Δloop (˜9.8 kDa), MC₇₂ (˜30 kDa), were expressed by inducing the T7 promoter of pRSETA-Eag I based vectors with isopropyl-β-D-thiogalactoside (IPTG) in E. coli C41(DE3) (Miroux & Walker, 1996 Over-production of proteins in Escherichia coli: Mutant hosts that allow synthesis of some membrane proteins and globular proteins at high levels. J. Mol. Biol. 260, 289-298) overnight at 25° C. and purified as described (Chatellier et al., 1998).

Proteins were analysed by electrospray mass spectrometry. Protein concentration was determined by absorbance at 276 nm using the method of Gill & von Hippel (1989 Calculation of protein extinction coefficients from amino acid sequence data. Anal. Biochem. 182, 319-326) and confirmed by quantitative amino acid analysis. In this study, protein concentrations refer to protomers, and not to oligomers.

Characterisation of MC₇₂

By both analytical size exclusion chromatography and analytical ultracentrifugation, both purified proteins, GroESΔloop and MC₇₂, were heptamers of seven 9.8 and seven 30 kDa subunits, respectively. The introduction into the GroES scaffold of a foreign polypeptide substantially larger than itself did not prevent oligomerisation. Electron microscopic studies of MC₇₂ revealed a diameter close to that of GroEL.

GroES Binding

The functionality of MC₇₂ was verified by examining GroES binding (followed by fluorescence) and mtMDH refolding.

### 11: Example 10

Reduction of Protein Aggregation in Huntingdon's Disease

Huntington's disease (HD), spinocerebellar ataxias types 1 and 3 (SCA1, SCA3), and spinobulbar muscular atrophy (SBMA) are caused by CAG/polyglutamine expansion mutations (Perutz, M. F. 1999 Trend Biochem. Sci. 24, 58-63; Rubinsztein, D. C. et al. 1999 J. Med. Genet. 36, 265-270). A feature of these diseases is ubiquitinated intraneuronal inclusions derived from the mutant proteins, which colocalize with heat shock proteins (HSPs) in SCA1 and SBMA and proteasomal components in SCA1, SCA3, and SBMA. Previous studies suggested that HSPs might protect against inclusion formation, because overexpression of HDJ-2/HSDJ (a human HSP40 homologue) reduced ataxin-1 (SCA1) and androgen receptor (SBMA) aggregate formation in HeLa cells (See Wyttenbach, A. et al. (2000) Proc. Natl. Acad. Sci. USA 97, 2899-2903).

These phenomena have been studied by transiently transfecting part of Huntingdon exon 1 in COS-7, PC12, and SH-SY5Y cells. Inclusion formation was not seen with constructs expressing 23 glutamines but was repeat length and time dependent for mutant constructs with 43-74 repeats. HSP70, HSP40, the 20S proteasome and ubiquitin colocalized with inclusions. Treatment with heat shock or with lactacystin, a proteasome inhibitor, increased the proportion of cells with inclusions of mutant Huntington exon 1. Thus, inclusion formation may be enhanced in polyglutamine diseases, if the pathological process results in proteasome inhibition or a heat-shock response. Overexpression of HDJ-2/HSDJ did not modify inclusion formation in PC12 and SH-SY5Y cells but increased inclusion formation in COS-7 cells. To our knowledge, this is the first report of an HSP increasing aggregation of an abnormally folded protein in mammalian cells and expands the current understanding of the roles of HDJ-2yHSDJ in protein folding (See Wyttenbach, A. et al. (2000) Proc. Natl. Acad. Sci. USA 97, 2899-2903).

In the eukaryotic cell, molecular chaperones might be involved in the actual formation of nuclear aggregates by stabilising the unfolded protein in an intermediate conformation which has the propensity to interact with neighbouring, unfolded proteins (Chirmer, E. C. & Lindquist, S. 1997 Proc. Natl. Acad. Sci. USA 94: 13932-7; DebBurnan, S. K. et al., 1997 Proc. Natl. Acad. Sci. USA 94: 13938-43; Welch, W. J. & Gambetti, P. 1998 Nature 392: 23-4). The chaperone's dual roles in aggregate formation and suppression may not be mutually exclusive, but rather dependent on the presence and level of chaperone expression. For example, the yeast chaperone Hsp104 (or bacterial GroEL) was shown to be necessary, at intermediate levels, for the propagation of the prion-like factor [PSI⁺], but when Hsp104 was overexpressed, [PSI⁺] was lost. Overexpression of the yeast homologue Hsp70 also inhibited [PSI⁺] (Chernoff, Y. O. et al., 1995 Science 268: 8804). A similar phenomenon may occur in spinocerebellar ataxia type 1, with endogenous levels of HDJ2/HDJ and/or Hsc70 contributing to the formation of ataxin-1 aggregates when the number of glutamine repeats is in the disease-causing range. As in yeast, it may be necessary to upregulate or modulate the level of molecular chaperones to reduce aggregate formation in affected neurons (Cummings, C. J. et al., 1998 Nat. Genet. 19: 148-54).

Recently, D. Rubinsztein et al. have shown that the overexpression of GroEL(191-345) minichaperone monomer reduced slightly but significantly the proportion of mutant Huntingdon exon 1-expressing PC12 and SH-SY5Y cells with inclusions and also reduced cell death. We have tested MC₇₂ in the same system.

The overexpression of MC₇₂ [i.e. the fusion protein GroESΔloop::GroEL(191-376)], like yeast Hsp104, reduced inclusion formation and cell death even further in the same cells. On the other hand, the overexpression of wild-type GroEL alone, whose activity is regulated by its co-chaperone GroES and ATP hydrolysis, had no effect.

The failure of Hsps to release their substrates in polyQ disease may be a common feature indicating the use of chaperones as therapeutic agents in these cases.

Then invention will now be further described by the following numbered paragraphs:

1. A polypeptide monomer capable of oligomerisation, said monomer comprising an heterologous amino acid or amino acid sequence inserted into the sequence of a subunit of an oligomerisable protein scaffold.

2. A polypeptide monomer according to paragraph 1, wherein the oligomerisable protein scaffold subunit it selected from the group consisting of bacteriophage T4 Gp31, Escherichia coli GroES and homologues thereof of the cpn10 family, and Escherichia coli GroEL and homologues thereof of the cpn60 family.

3. A polypeptide monomer according to paragraph 1 or paragraph 2, wherein the heterologous amino acid or amino acid sequence is inserted into the sequence of the oligomerisable protein scaffold subunit such that both the N and C termini of the polypeptide monomer are formed by the sequence of the oligomerisable protein scaffold subunit.

4. A polypeptide monomer according to any preceding paragraph, wherein the heterologous amino acid or amino acid sequence is inserted into the oligomerisable protein scaffold subunit by replacing one or more amino acids thereof.

5. A polypeptide monomer according to paragraph 4, wherein the oligomerisable protein scaffold subunit is bacteriophage T4 Gp31 and the heterologous amino acid or amino acid sequence is inserted into the oligomerisable protein scaffold subunit by substantially replacing the mobile loop between amino acid positions 23 and 44.

6. A polypeptide monomer according to paragraph 4, wherein the oligomerisable protein scaffold subunit is Escherichia coli GroES and the heterotogous amino acid or amino acid sequence is inserted into the oligomerisable protein scaffold subunit by substantially replacing the mobile loop between amino acid positions 15 and 34.

7. A polypeptide monomer according to paragraph 4, wherein the oligomerisable protein scaffold subunit is bacteriophage T4 Gp31 and the heterologous amino acid or amino acid sequence is inserted between positions 54 and 67 of the oligomerisable protein scaffold subunit.

8. A polypeptide monomer according to paragraph 4, wherein the oligomerisable protein scaffold subunit is Escherichia coli GroES and the heterologous amino acid or amino acid sequence is inserted between positions 43 and 63 of the oligomerisable protein scaffold subunit.

9. A polypeptide monomer according to paragraph 4, wherein the oligomerisable protein scaffold subunit is bacteriophage T4 Gp31 and heterologous amino acids or amino acid sequences are inserted at both positions described in paragraphs 5 and 7.

10. A polypeptide monomer according to paragraph 4, wherein the oligomerisable protein scaffold subunit is Escherichia coli GroES and heterologous amino acids or amino acid sequences are inserted at both positions described in paragraphs 6 and 8.

11. A polypeptide monomer according to paragraph 2, wherein the heterologous amino acid sequence is displayed at the N or C terminus of the oligomerisable protein scaffold subunit.

12. A polypeptide oligomer comprising two or more polypeptide monomers according to any preceding paragraph.

13. A polypeptide oligomer according to paragraph 12, which is a homooligomer.

14. A polypeptide oligomer according to paragraph 12, which is a heterooligomer.

15. A polypeptide oligomer according to paragraph 14, wherein complementary biological activities are juxtaposed through the oligomerisation of different polypeptide monomers.

16. A polypeptide oligomer according to any one of paragraphs 12 to 15, wherein the monomers are covalently crosslinked.

17. A polypeptide oligomer according to any one of paragraphs 12 to 16, wherein the protein scaffold is in the form of a ring.

18. A polypeptide oligomer according to paragraph 17, wherein the ring is a heptameric ring.

19. A polypeptide monomer or oligomer according to any preceding paragraph, wherein the heterologous amino acid sequence is an immunogen.

20. A polypeptide oligomer or monomer according to paragraph 19, wherein the protein scaffold is bacterial or bacteriophage in origin.

21. A polypeptide according to paragraph 19 or paragraph 20, for use in the detection or neutralisation of antibodies in vivo.

22. Use of a polypeptide according to paragraph 19 or paragraph 20 for the detection or neutralisation of antibodies in vitro.

23. A polypeptide according to paragraph 19 or paragraph 20, for use as a vaccine.

24. A polypeptide monomer or oligomer according to any one of paragraphs 1 to 18, wherein the heterologous amino acid sequence is an antibody or an antigen-binding fragment thereof.

25. A polypeptide according to paragraph 24, wherein the antibody fragment is a natural or camelised VH domain.

26. A polypeptide according to paragraph 25, wherein the antibody fragment is a VH CDR3.

27. A polypeptide according to any one of paragraphs 24 to 26, for use in the detection or neutralisation of antigens in vivo.

28. Use of a polypeptide according to any one of paragraphs 24 to 26 for the detection or neutralisation of antigens in vitro.

29. A polypeptide monomer or oligomer according to any one of paragraphs 1 to 18, wherein the heterologous amino acid sequence is a ligand for a receptor.

30. A polypeptide monomer or oligomer according to any one of paragraphs 1 to 18, wherein the heterologous amino acid sequence is a substrate for a kinase or phosphatase.

31. A polypeptide according to paragraph 29 or paragraph 30, wherein the heterologous amino acid sequence comprises an at least partly randomised portion.

32. A polypeptide monomer or oligomer according to any one of paragraphs 1 to 18, wherein the heterologous amino acid sequence is capable of mediating a biological activity.

33. A polypeptide according to paragraph 32, wherein the heterologous amino acid sequence is selected from the group consisting of an enzyme, an antibiotic, an enzyme inhibitor, a molecule involved in cell signalling, a hormone, an antigen, an immunogen, a nuclear localisation sequence, a cellular uptake sequence, a DNA binding sequence, a solid surface binding sequence composed of random charged amino acids, a receptor and a ligand for a receptor.

34. A polypeptide oligomer according to paragraph 32 or 33, which comprises two or more different heterologous amino acid sequences having different biological activities.

35. A nucleic acid sequence encoding a polypeptide according to any preceding paragraph.

36. A method for preparing a polypeptide according to any one of paragraphs 1 to 35, comprising the steps of inserting a nucleic acid sequence encoding a heterologous polypeptide into a nucleic acid sequence encoding a subunit of an oligomerisable protein scaffold, incorporating the resulting nucleic acid into an expression vector, and expressing the nucleic acid to produce the polypeptide monomers.
